(19) United States
(12) Patent Application Publication
(10) Pub. No.: US 2026/0191738 A1
(43) Pub. Date: Jul. 9, 2026

(54) MEDICAL SENSOR DATA TRANSFER CABLES

(71) Applicant: ZOLL Medical Corporation, Chelmsford, MA (US)

(72) Inventors: Suzanne Crowell, Beverly, MA (US); Dan E. Goldman, Shrewsbury, MA (US); Frederick K. Newey, Pelham, NH (US); Mark Weary, Billerica, MA (US); Lisa M. Campana, Waltham, MA (US)

(73) Assignee: ZOLL Medical Corporation, Chelmsford, MA (US)

(21) Appl. No.: 19/115,551

(22) PCT Filed: Sep. 27, 2023
(86) PCT No.: PCT/US2023/033850

§ 371 Date: Mar. 26, 2025

Related U.S. Application Data
(60) Provisional application No. 63/410,959, filed on Sep. 28, 2022.

(51) Int. Cl. A61H 31/00; A61M 16/00; A61M 16/06

(52) U.S. Cl. CPC A61H 31/005; A61M 16/0084; A61M 16/06; A61M 2016/0033; A61M 2205/502; A61M 2205/583; A61M 2205/587; A61M 2230/30

(57) ABSTRACT

In an illustrative embodiment, a data transfer cable for transferring sensor data between a sensor device and a medical device includes at least one illumination element configured to provide at least two visual indications visible in a location distal to the connector, each visual indication corresponding to a respective state of the cable or a sensor connected thereto. The cable may include processing circuitry configured to receive, from the medical device responsive to user interaction with a portion of a user interface corresponding to the sensor connected to the cable, an identify signal, and provide a first visual indication for indicating correspondence between the cable and the sensor connected thereto. A second visual indication may provide prompting/feedback to a caregiver while using the sensor device.

### RELATED APPLICATIONS

This application claims priority to U.S. Provisional Patent Application Ser. No. 63/410,959 filed Sep. 28, 2022.

This application is related to U.S. patent application Ser. No. 17/211,900, entitled “Medical Device System and Hardware for Sensor Data Acquisition” filed Mar. 25, 2021. All above identified applications are hereby incorporated by reference in their entireties.

### BACKGROUND

Medical devices such as patient monitors and defibrillators obtain physiological and medical treatment data via sensors. For example, physiological data may include patient data such as vital signs, electrocardiograms (ECGs), pulse oximetry data, and/or capnography data. Medical treatment data may include treatment administration metrics such as cardiopulmonary resuscitation (CPR) parameters. Sensors configured to provide this data may couple to the medical devices via cable connections to data interface ports. The data interface ports capture sensor data and provide this captured data to the medical device for analysis and display.

### SUMMARY OF ILLUSTRATIVE EMBODIMENTS

In one aspect, the present disclosure relates to a data transfer cable for transferring sensor data between a sensor and a medical device, the data transfer cable including an insulative sheath surrounding a number of conductive wires, a medical device connector for connecting the data transfer cable to a data port of a medical device, and at least one illumination element in electrical communication with at least one wire of the number of conductive wires, the at least one illumination element being configured to provide at least two visual indications visible in a location distal to the medical device connector, where each visual indication of the at least two visual indications corresponds to a respective state of the data transfer cable or a sensor connected thereto. The data transfer cable may include processing circuitry configured to receive, from the medical device responsive to user interaction with a portion of a user interface of the medical device corresponding to the sensor connected to the data transfer cable, an identify signal, and responsive to receiving the identify signal, cause a first illumination element of the at least one illumination element to provide a first visual indication of the at least two visual indications for indicating correspondence between the data transfer cable and the sensor connected thereto.

In some embodiments, the data transfer cable includes an isolation device configured to limit current flow leakage between the medical device and the sensor. The isolation device may be configured to transfer power across an isolation barrier unidirectionally toward the processing circuitry and transmit communication signals bi-directionally across the isolation barrier. The data transfer cable may include a noise shield disposed between the isolation device and the processing circuitry.

In some embodiments, the sensor is one of an invasive blood pressure sensor, a non-invasive blood pressure sensor, a temperature sensor, a pulse oximetry sensor, a capnography sensor, or an airway flow sensor. The sensor may be one of a breath sounds sensor, a heart sounds sensor, a lung sounds sensor, a dual shock defibrillator sensor, an electroencephalogram (EEG) sensor, or a glucose monitoring sensor. The sensor may be an electrocardiogramsor or an extended ECG sensor. The at least one illumination element may include a respective illumination element corresponding to each ECG contact of a number of ECG contacts configured for individual positioning upon a patient. The user interaction may include interaction with a selected ECG signal graph of a number of ECG signal graphs presented on a display of the medical device, and causing the first illumination element to provide the first visual indication includes causing illumination corresponding to a set of ECG contacts of the number of ECG contacts contributing to the selected ECG signal graph.

In some embodiments, the data transfer cable includes a housing disposed along the insulative sheath or at a proximal end of the insulative sheath opposite the medical device connector, where the housing includes the processing circuitry and the at least one illumination element. The housing may include the sensor. The housing may include, at an end opposite the insulative sheath, a sensor connector for releasably engaging a mating connector of a sensor device including the sensor.

In some embodiments, the sensor includes an airway flow sensor, and the processing circuitry is configured to receive, from the medical device, timing signals corresponding to delivery of airflow to a patient, and, using the timing signals, cause one or more illumination elements of the at least one illumination element to provide a second visual indication of the at least two visual indications for prompting airflow delivery by a caregiver. A ventilation system including a bag valve mask may include the airway flow sensor. A ventilation system may include or be connected with the one or more illumination elements. And the one or more illumination elements may be arranged such that the second visual indication can be recognized by a caregiver observing the one or more illumination elements from a number of orientations. The one or more illumination elements may be arranged on a three-dimensional protrusion connected to a housing of the ventilation system. The one or more illumination elements may be arranged on a pivoting attachment of the ventilation system. The one or more illumination elements may be arranged on a rotating attachment of the ventilation system. The sensor may include an airway flow sensor, and the processing circuitry may be configured to receive, from the medical device, feedback signals corresponding to at least one of a timing and a volume of delivery of airflow to a patient, and using the feedback signals, cause one or more illumination elements of the at least one illumination element to provide a second visual indication of the at least two visual indications for providing feedback to a caregiver regarding airflow delivery. The second visual indication may visually mimic corresponding visual feedback presented in a region of a display of the medical device. The second visual indication may include at least one of a numeric rate indication or a numeric volume indication. A digital display may include the at least one illumination element, and the housing includes an aperture for the display. The digital display may be a liquid crystal display (LCD) or a light emitting diode (LED) display. The at least one illumination element may include at least one light emitting diode (LED), and the housing may include at least one translucent region disposed proximate each LED of the at least one LED. A first LED of the at least one LED may be a multi-color LED, and the at least two visual indications may include a first color indication of the multi-color LED and a second color indication of the multi-color LED.

In some embodiments, the portion of the user interface of the medical device is a portion of a display of the medical device. The display of the medical device may be a touch display.

In some embodiments, the data transfer cable includes, at an end opposite the medical device connector, a sensor connector for releasably engaging a mating connector of a sensor device including the sensor. The sensor connector may be configured to releasably engage one of a set of sensor devices, each sensor device including a different type of sensor. The processing circuitry may be configured to format sensor data from the respective type of sensor of each sensor device of the set of sensor devices into a sensor-agnostic data format accepted by the medical device.

In some embodiments, the processing circuitry is further configured to receive, from the medical device after connection of the medical device connector to the medical device, a connection signal, and responsive to receiving the connection signal, cause one or more illumination elements of the at least one illumination element to provide a second visual indication of the at least two visual indications for indicating connection between the medical device and the data transfer cable. The second visual indication may remain illuminated while the data transfer cable is connected to the medical device. The one or more illumination elements may include the first illumination element. The connection signal may indicate that a data transfer connection with the data transfer cable has been authenticated by the medical device. The processing circuitry may be further configured to engage in an authentication handshake with the medical device.

In some embodiments, the processing circuitry is further configured to receive at least one sensor signal from the sensor, and responsive to receiving the at least one sensor signal, cause a second illumination element of the at least one illumination element to provide a second visual indication of the at least two visual indications for indicating data connection between the data transfer cable and the sensor. The second illumination element may be different than the first illumination element. The second visual indication may remain illuminated while the processing circuitry is communicating with the sensor.

In some embodiments, the sensor includes a cardiopulmonary resuscitation (CPR) compression sensor, and the processing circuitry is configured to receive, from the medical device, timing signals corresponding to delivery of compressions to a patient, and using the timing signals, cause one or more illumination elements of the at least one illumination element to provide a second visual indication of the at least two visual indications for prompting compression delivery by a caregiver.

In some embodiments, the sensor includes a cardiopulmonary resuscitation (CPR) compression sensor, and the processing circuitry is configured to receive, from the medical device, feedback signals corresponding to at least one of a timing and a depth of compression delivery to a patient, and using the feedback signals, cause one or more illumination elements of the at least one illumination element to provide a second visual indication of the at least two visual indications for providing feedback to a caregiver regarding compression delivery. The second visual indication may visually mimic corresponding visual feedback presented in a region of a display of the medical device. The second visual indication may include at least one of a numeric rate indication or a numeric depth indication.

In some embodiments, the sensor is an invasive blood pressure (IBP) sensor, and the processing circuitry is configured to receive, from the medical device, a zeroing signal corresponding to zeroing an IBP probe including the IBP sensor, and, responsive to the zeroing signal, cause one or more illumination elements of the at least one illumination element to provide a second visual indication of the at least two visual indications for identifying the IBP probe is being zeroed. The processing circuitry may be configured to cause one or more illumination elements of the at least one illumination element to provide a third visual indication of the at least two visual indications for prompting a user to zero the probe.

In one aspect, the present disclosure relates to patient monitoring and treatment system for providing sensor data capture capabilities, the system including a medical device including a display, at least one data interface port configured to enable power transfer to a sensor unit and data communications between at least one sensor of the sensor unit and the medical device, and medical device processing circuitry configured to analyze sensor signals received via the at least one data interface port and present information corresponding to the sensor signals upon the display. The patient monitoring and treatment system may include a data transfer cable configured to matingly connect to the at least one data interface port, the data transfer cable including an insulative sheath surrounding a number of conductive wires, a medical device connector for connecting the data transfer cable to a given data interface port of the at least one data interface port of the medical device, at least one illumination element in electrical communication with at least one wire of the number of conductive wires, the at least one illumination element disposed in a location distal to the medical device connector, and cable processing circuitry configured to enable communication between the medical device and the sensor unit and to enable feedback to a user via the at least one illumination element. The medical device processing circuitry may be further configured to coordinate visual feedback presentations to the user via the display of the medical device and the at least one illumination element of the data transfer cable.

In some embodiments, coordinating visual feedback presentations includes causing presentation, at both the display of the medical device and at the at least one illumination element, of coaching feedback for using the sensor unit. The coaching feedback may visually mimic corresponding visual feedback presented in a region of the display of the medical device. The sensor unit is an invasive blood pressure (IBP) probe including an IBP sensor, and the coaching feedback includes feedback for setting up the IBP probe. Coordinating the visual feedback presentations may include presenting, on the display, a prompt for zeroing the IBP probe, and providing, to the medical device processing circuitry, instructions to provide visual feedback, via the at least one illumination element, corresponding to initiation of zeroing the IBP probe. Coordinating the visual feedback presentations may include presenting, on the display, a prompt for identifying a use case for the IBP probe, and providing, to the cable processing circuitry, instructions to provide visual feedback, via the at least one illumination element, corresponding to selection of the use case for the IBP probe. The use case may be one of invasive blood pressure (IPB), arterial blood pressure (ART), pulmonary artery pressure (PAP), central venous pressure (CVP), or intra-cranial pressure (ICP).

In some embodiments, the sensor unit is a ventilation unit including an airflow sensor, and the coaching feedback includes feedback for delivering a target volume of air to a patient. The cable processing circuitry may be configured to receive, from the medical device, timing signals corresponding to delivery of airflow to a patient, and using the timing signals, cause one or more illumination elements of the at least one illumination element to present the coaching feedback for airflow delivery by a caregiver. A bag valve mask may include the at least one illumination element. The one or more illumination elements may be arranged such that the coaching feedback can be recognized by a caregiver observing the bag valve mask from a number of orientations. The one or more illumination elements may be arranged on a three-dimensional protrusion of the bag valve mask. The one or more illumination elements may be arranged on a pivoting attachment of the bag valve mask. The one or more illumination elements may be arranged on a rotating attachment of the bag valve mask. The coaching feedback may include at least one of a numeric rate indication or a numeric volume indication. The system may include a second data transfer cable including a second insulative sheath surrounding a number of second conductive wires, a second medical device connector for connecting the second data transfer cable to another given data interface port of the at least one data interface port of the medical device, at least one second illumination element in electrical communication with at least one wire of the number of second conductive wires, the at least one second illumination element being visible in a location distal to the second medical device connector, and second cable processing circuitry configured to enable communication between the medical device and a chest compression sensor unit and to enable feedback to a second user via the at least one second illumination element. The medical device processing circuitry may be configured to coordinate ventilation feedback to the user and compression feedback to the second user via the at least one illumination element of the data transfer cable and the at least one second illumination element of the second data transfer cable. Coordinating the ventilation feedback and the compression feedback may include coordinating prompting for ventilation timing with chest compression timing.

In some embodiments, the sensor includes a cardiopulmonary resuscitation (CPR) compression sensor, and the cable processing circuitry is configured to receive, from the medical device, timing signals corresponding to delivery of compressions to a patient, and using the timing signals, cause one or more illumination elements of the at least one illumination element to present the coaching feedback for prompting compression delivery by a caregiver. The cable processing circuitry may be configured to receive, from the medical device, feedback signals corresponding to at least one of a timing or a depth of compression delivery to a patient, and, using the feedback signals, cause one or more illumination elements of the at least one illumination element to present a level of sufficiency of the at least one of the timing and the depth of the compression delivery. Presenting the level of sufficiency may include presenting at least one of a numeric rate indication or a numeric depth indication. The system may include a second data transfer cable including a second insulative sheath surrounding a number of second conductive wires, a second medical device connector for connecting the second data transfer cable to another given data interface port of the at least one data interface port of the medical device, at least one second illumination element in electrical communication with at least one wire of the number of second conductive wires, the at least one second illumination element being visible in a location distal to the second medical device connector, and second cable processing circuitry configured to enable communication between the medical device and a ventilation sensor unit and to enable feedback to a second user via the at least one second illumination element. The medical device processing circuitry may be configured to coordinate CPR feedback to the user and ventilation feedback to the second user via the at least one illumination element of the data transfer cable and the at least one second illumination element of the second data transfer cable. Coordinating the CPR feedback and the ventilation feedback may include coordinating prompting for chest compression timing with ventilation timing.

In some embodiments, the display includes a touch-sensitive interface, and the medical device includes a lock control for disabling the touch-sensitive interface. Responsive to disabling the touch-sensitive interface via actuation of the lock control, the medical device processing circuitry may be configured to present, upon the display, a lock enabled indicator. The medical device may include a manual navigation control for navigating and interacting with contents of the display. Navigating and interacting via the manual navigation control may be disabled while the lock control is in a disabled position.

In one aspect, the present disclosure relates to a ventilation sensor unit including a housing including an airflow sensor, an airflow delivery element for manually controlling airflow delivery to a patient, a data transfer cable extending from the housing, the data transfer cable including an insulative sheath surrounding a number of conductive wires, and a medical device connector for connecting the data transfer cable to a data port of a medical device, at least one illumination element in electrical communication with at least one wire of the number of conductive wires, and processing circuitry configured to receive, from the medical device, timing signals corresponding to delivery of airflow to a patient, using the timing signals, cause one or more illumination elements of the at least one illumination element to provide a visual indication for prompting airflow delivery by a caregiver, and provide, to the medical device via the data transfer cable, at least one airflow signal from the airflow sensor indicative of at least one of a rate or a volume of airflow delivered to the patient.

In some embodiments, a bag valve mask includes the housing. The one or more illumination elements may be arranged such that the second visual indication can be recognized by a caregiver observing the housing from a number of orientations. The one or more illumination elements may be arranged on at least one surface of the housing. The one or more illumination elements may include a number of LED elements arranged in a ring formation on the housing. The number of LED elements may include multi-colored LED elements. The one or more illumination elements may be arranged to provide a digital display configured to present numeric feedback to the caregiver. The numeric feedback may include at least one of a volume or a rate.

In some embodiments, the one or more illumination elements are arranged on a three-dimensional protrusion of the housing. The one or more illumination elements may be arranged on a pivoting attachment of the housing. The one or more illumination elements may be arranged on a rotating attachment of the housing.

In some embodiments, the processing circuitry is further configured to receive, from the medical device, feedback signals corresponding to at least one of a timing and a volume of recent delivery of airflow to a patient, and, using the feedback signals, cause one or more illumination elements of the at least one illumination element to provide sufficiency feedback to a caregiver regarding sufficiency of airflow delivery. The sufficiency feedback may visually mimic corresponding visual feedback presented in a region of a display of the medical device. The sufficiency feedback may include at least one of a numeric rate indication or a numeric volume indication. The sufficiency feedback may include a respective color of a set of colors corresponding to a target range and outside of the target range.

In one aspect, the present disclosure relates to a system for monitoring invasive blood pressure (IBP) in a patient, the system including an invasive blood pressure (IBP) probe including a housing and an invasive blood pressure (IBP) sensor, a data transfer cable extending from the IBP probe, the data transfer cable including an insulative sheath surrounding a number of conductive wires, and a medical device connector for connecting the data transfer cable to a data port of a medical device. The medical device may include the data port, a display, and processing circuitry configured to recognize insertion of the data transfer cable in the data port, present, on the display, a prompt for zeroing the IBP probe, and responsive to input to the medical device by a caregiver, initiate a zeroing process with the IBP probe.

In some embodiments, the processing circuitry of the medical device is configured to present, to the caregiver, a prompt on the display of the medical device to one of a set of use cases for the IBP probe. The set of use cases may include two or more of invasive blood pressure (IBP), arterial blood pressure (ART), pulmonary artery pressure (PAP), central venous pressure (CVP), or intra-cranial pressure (ICP). Responsive to the caregiver entering a selected use case of the set of use cases, the processing circuitry of the medical device may format a section of the display to present metrics related to the IBP probe in a format corresponding to the selected use case.

In some embodiments, the IBP probe further includes at least one illumination element in electrical communication with at least one wire of the number of conductive wires, and probe processing circuitry configured to cause presentation of visual feedback to the caregiver via the at least one illumination element. The processing circuitry of the medical device may be configured to detect interaction with a region of the display presenting metrics related to the IBP probe, and responsive to the detecting, issue a signal via to the probe processing circuitry of the IBP probe to cause illumination of one or more illumination elements of the at least one illumination element. The processing circuitry of the medical device may be further configured to provide, to the data transfer cable processing circuitry, instructions to provide visual feedback, via the at least one illumination element, corresponding to initiation of zeroing the IBP probe.

In some embodiments, the IBP probe further includes probe processing circuitry configured to provide authentication information to the processing circuitry of the medical device, and the processing circuitry of the medical device is configured, after recognizing insertion of the data transfer cable in the data port, initiate an authentication sequence with the probe processing circuitry.

In some embodiments, the data transfer cable further includes data transfer cable processing circuitry configured to provide authentication information to the processing circuitry of the medical device, and the processing circuitry of the medical device is configured to, after recognizing insertion of the data transfer cable in the data port, initiate an authentication sequence with the data transfer cable processing circuitry. The IBP probe may be releasably attachable to the data transfer cable. The IBP probe may be one type of a number of types of sensor devices compatible to releasably attach to the data transfer cable.

In some embodiments, the data transfer cable further includes at least one illumination element in electrical communication with at least one wire of the number of conductive wires, and data transfer cable processing circuitry configured to cause presentation of visual feedback to the caregiver via the at least one illumination element. The processing circuitry of the medical device may be further configured to provide, to the data transfer cable processing circuitry, instructions to provide visual feedback, via the at least one illumination element, corresponding to initiation of zeroing the IBP probe. The processing circuitry of the data transfer cable may be further configured to receive, from the medical device, a zeroing signal corresponding to zeroing an IBP probe including the IBP sensor, and, responsive to the zeroing signal, cause one or more illumination elements of the at least one illumination element to provide a second visual indication of the at least two visual indications for identifying the IBP probe is being zeroed. The IBP probe may be releasably attachable to the data transfer cable. The IBP probe may be one type of a number of types of sensor devices compatible to releasably attach to the data transfer cable.

The foregoing general description of the illustrative implementations and the following detailed description thereof are merely exemplary aspects of the teachings of this disclosure and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A and FIG. 1B illustrate data cable state feedback mechanisms in an example system including a medical device configured with multiple sensor-agnostic data interface ports and a data transfer cable connected thereto;

FIG. 1C illustrates an example touch screen lock configuration of the medical device of FIG. 1A and FIG. 1B;

FIG. 2A through FIG. 2C illustrate a series of stages of an invasive blood pressure (IBP) sensor setup routine;

FIG. 3 is a flow chart of an example method for automatically initializing an invasive blood pressure probe;

FIG. 4A, FIG. 4B-1 through FIG. 4B-4, 4C, and 4D illustrate example user interface output of a medical device having a ventilation system connected via a data transfer cable;

FIG. 5A illustrates example user interface output coordinated between a display region of a ventilation system and a medical device the ventilation system is connected to;

FIG. 5B illustrates example user interface output coordinated between a display region of a medical device and a set of ECG electrodes;

FIG. 6A through FIG. 6E illustrate a sensor-agnostic data interface (SA-DI) cable coupled to different types of sensors or sensor devices and example feedback corresponding to each;

FIG. 7A, FIG. 7B-1, and FIG. 7B-2 illustrate a flow sensor system including an illuminated user interface for providing coaching and/or feedback to a caregiver supplying ventilation to a patient;

FIG. 8A and FIG. 8B illustrate a flow chart of an example method for coordinating caregiver feedback between a computing device and one or more sensor devices connected to a medical device;

FIG. 9 illustrates a schematic example of information available from an authentication circuit and a cable memory;

FIG. 10 illustrates a schematic diagram of an example medical device/data transfer cable system;

FIG. 11 illustrates a schematic diagram of an example medical device having a removable sensor hub; and

FIG. 12 illustrates a block diagram of components an example sensor data gathering device and patient interface device.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,”“inner,”“outer,” and the like that may be used herein merely describe points of reference and do not necessarily limit embodiments of the present disclosure to any particular orientation or configuration.

Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

During a medical event, a medical device may be used by a caregiver (e.g., a first responder, a paramedic, a physician, a nurse, a rescue worker, etc.) to provide medical therapy to a patient and/or may be used to monitor the patient. The medical device may be, for example, a patient monitor, a therapeutic medical device (e.g., a defibrillator, an automated compression device, a ventilator, etc.), a therapeutic medical device/patient monitor, or a modular therapeutic medical device/patient monitor. These types of medical devices are examples only and other types and combinations of medical devices are within the scope of the disclosure.

The medical device may be configured to couple to one or more sensors. The sensors may include one or more combined therapy delivery/sensing components such as defibrillation electrodes configured to sense and monitor a patient's electrocardiogram (ECG) and to deliver electrotherapy. The medical device may collect data via the one or more sensors. The data may include physiological sensor data and/or medical or resuscitative treatment data. The physiological sensor data may include, for example, invasive blood pressure (IBP), non-invasive blood pressure (NIBP), electrocardiogram (ECG) data, pulse oximetry data (SpO2), capnography data, methemoglobin (SpMet), hemoglobin, body temperature, cerebral oxygen saturation (rSO2), heart rate, and/or other vital signs. The physiological data may also include imaging data such as, for example, laryngoscopy and/or ultrasound. The medical or resuscitative treatment data may include, for example, CPR performance data derived from measurements obtained from a chest compression sensor (e.g., compression depth, compression rate, chest release, perfusion performance, etc.) and/or ventilation data from measurements obtained from an airway flow sensor (e.g., ventilation tidal volume, ventilation rate, ventilation minute volume, ventilation performance, etc.). These types of data are examples only and not limiting of the disclosure and are discussed in further detail below.

The medical device and the one or more sensors may couple to one another via a data transfer cable. To enable these wired couplings, the medical device may include one or more data interface (DI) ports. Each data interface port may be configured to removably couple to a data transfer cable, the data transfer cable coupled, in turn, to a sensor. The DI port may be a sensor-specific DI (SS-DI) port. For example, in various implementations, the sensor-specific DI port may be configured with a cable contact count and wiring assignments, voltage(s), and/or processor configuration for signal processing protocol compatible with one type of sensor but incompatible with another type of sensor. For example, the sensor-specific DI port may be compatible with an invasive blood pressure (IBP) accessory (e.g., a sensor/data transfer cable combination for IBP) but may not be compatible with a capnography accessory (e.g., sensor/data transfer cable combination for sensing exhaled gas flow such as carbon dioxide). As an incompatible accessory, the capnography accessory may not mate physically with the IBP specific port and/or may not be electrically compatible and/or may provide sensing data to the IBP specific port that the IBP specific port cannot process due to differences in data protocols for different sensor data types. The DI port may be a sensor-agnostic DI (SA-DI) port configured to capture a variety of sensor data types, each provided via a data transfer cable configured to provide the sensor data in a format compatible with the sensor-agnostic DI port. Thus, the caregiver can attach any SA-DI compatible sensor cable to any available sensor-agnostic DI port. The data transfer cable may include hardware compatible with the cable contact count and wiring assignments and voltage(s) of the sensor-agnostic DI port. Furthermore, the data transfer cable may include a processor, stored software, and patient leakage current isolation that provide the sensor data in an agnostic format, throttle agnostic power delivery according to the needs of the sensor, and tailor the patient leakage current isolation to the specific sensor power requirements.

In some implementations, a data transfer cable includes one or more illumination elements disposed on the cable, on a housing coupled to the cable, and/or on an electromechanical connector configured to connect a sensor device to the data transfer cable. The illumination elements may have different operating modes to indicate different state information related to the data transfer cable and/or a sensor coupled thereto, such as, in some examples, a power on state, a power off state, an authentication state, a sensor fault state, a sensor working state, a sensor type, and/or a communication state. The illumination elements may indicate state information, in some examples, based on color, continuity of illumination (e.g., blinking or continuous or various rates of blinking), and/or an absence of illumination. In some embodiments, the data transfer cable includes a low light sensor electrically coupled to the at least one illumination element and configured to disable illumination under low light conditions.

In some implementations, one or more illumination elements of a data transfer cable provide infrared illumination. For example, the wavelength of the infrared illumination may be in the range of 900-1750 nm. The infrared illumination may provide an advantage in military settings by enabling identification of the sensor, for example with night vision goggles.

As discussed above, a medical device, such as a patient monitor or patient monitor/defibrillator, often includes an attachment of multiple sensors for effective patient care. Each sensor may be connected to the medical device via a wired cable that requires a DI port. For the sensor-agnostic DI ports, a number of compatible and, therefore potentially visually indistinguishable (at least from a port-end of the cable) cables may be leading from the medical device. Further, these cables may be crossing, overlapping, or otherwise obscuring visibility to which cable is connected to which sensor device. For example, the medical device may provide three DI ports to enable connection to an IBP sensor, a temperature sensor, and an airway flow sensor.

To improve ease of interpretation of a display region of a medical device presenting information related to multiple data transfer cables, in some implementations, one or more illumination elements of the data transfer cable may illuminate in response to user input at the medical device. The user input, for example, may allow the user to uniquely match a particular display region (e.g., data output) with a particular cable/sensor, providing the advantage of swift recognition of which data transfer cable/sensor device combination is associated with which output. For example, the medical device may include a button and/or touch screen control configured to accept a user inquiry to identify a sensor type on a data transfer cable. The host processor of the medical device may cause one or more illumination elements of the data transfer cable to illuminate in response to the user input at the medical device. The host processor of the medical device, for example, may cause the illumination element(s) to illuminate in response to a user touch and/or other user input (e.g., a cursor or other selectable screen indicator) to the display of the medical device.

In some implementations, the data transfer cable includes a cable processor configured to present information to a caregiver via the illumination element(s). In such implementations, the host processor of the medical device may issue commands to the cable processor of the data transfer cable to cause illumination in response to the user input.

In some implementations, the data transfer cable includes an identification contact/wire combination for cable identification queries. The host processor of the medical device may provide a signal to the illumination element(s) via this identification contact/wire combination.

In some implementations, data transfer cables include a cable user interface including a set of illumination elements or illuminated display configured to present caregiver feedback and/or prompting. While prompting and/or feedback information may be presented at a display of the medical device, this display may be remote from a caregiver or otherwise awkward for a caregiver to view. To overcome this disadvantage, a data transfer cable user interface may be positioned or disposed along the cable or at the electromechanical connector interface with the sensor device in proximity to a caregiver providing care to a patient. With the prompting and/or feedback information provided directly at the point of care via the data transfer cable user interface, the caregiver does not need to turn his/her head to observe a screen that may be located or otherwise positioned at an inconvenient or uncomfortable position for viewing. The user output may depend on the type of sensor coupled to the data transfer cable. For example, a data transfer cable with an airway flow sensor may present an airway flow sensor user interface. The airway flow sensor user interface, for example, may provide bag valve mask feedback for coaching and/or prompting a user to administer ventilations according to predetermined ventilation targets, such as within predetermined range(s) for ventilation tidal volume, ventilation rate and/or ventilation minute volume. In another example, a data transfer cable with a CPR compression sensor may include a CPR compression sensor user interface for prompting and/or coaching the user to administer chest compressions according to predetermined chest compression targets, such as within predetermined range(s) for chest compression depth, chest compression rate, and/or release velocity. The prompts presented at the user interface, for example, may include numerical values that illustrate the respective ventilation/compression parameters. In addition, or alternatively, the user interface may provide instructive feedback for the user if the ventilation and/or compression parameters are outside of the desired target range. For example, if the ventilation tidal volume is outside of the desired target range, then a portion of the user interface may provide a visual indication (e.g., highlighting the numerical value representing tidal volume, illuminating a background of the numerical value in a warning color such as yellow, orange or red, illuminating the numerical value itself in a warning color such as yellow, orange or red, etc.) that the user should pay attention to how much tidal volume is being administered to the patient. Similarly, if the ventilation rate is outside of the desired target range, then a portion of the user interface may provide a visual indication (e.g., highlighting the numerical value representing ventilation rate, illuminating a background of the numerical value in a warning color, illuminating the numerical value itself in a warning color, etc.) that the user should adjust how fast or slow ventilations are being administered. In some embodiments, when the compression depth is outside of the desired target range, then the user interface may indicate (e.g., highlight of the numerical value representing compression depth, illuminating a background of the numerical value in a warning color, illuminating the numerical value itself in a warning color, etc.) that the user should adjust the depth of chest compressions given. And when the compression rate falls outside of the desired target range, then the user interface may similarly indicate (e.g., highlight the numerical value representing compression rate, illuminating a background of the numerical value in a warning color, illuminating the numerical value itself in a warning color, etc.) that the user is outside of the target compression rate range.

In some implementations, the medical device includes one or more removable sensor hubs that provide one or more DI ports. These DI ports may be sensor-agnostic, sensor-specific, or a combination thereof. The removable sensor hub may also include hardware and/or software controls for a particular sensor or combination of sensors. For example, the sensor hub may provide pneumatic controls and a pump system for NIBP and/or capnography. The one or more removable sensor hubs may removably couple to the medical device within a medical device housing or to an exterior of the medical device housing. The sensor hub may capture sensor data when physically coupled and when physically uncoupled from the medical device. The removable sensor hub may communicatively couple to the medical device via a wired or a wireless connection.

When using removable sensor hubs, a total number of cables communicatively connected to a medical device may be increased. For example, the medical device may include a display divided into a multitude of display regions on the screen, each region devoted to displaying sensor data and/or status indications regarding a given sensor connected to the medical device directly and/or via a sensor hub. To clarify interpretation of the various data, systems and methods described herein provide the opportunity to visually connect a particular cable with a particular display region through user selection of the display region. For example, touching a section of the display screen of the medical device may cause illumination on the cable distal to the medical device.

Additionally, a caregiver can place the sensor hub on a gurney or other supportive item near the patient and in a location easily viewable and accessible for the caregiver. In an implementation, the sensor hub may be sufficiently lightweight to place on the patient without any negative impact on patient care and/or resuscitation. Locating the sensor hub as close to the patient as possible, including possibly on the patient, may enable the caregiver to provide care and view a data display on the sensor hub and/or connect or disconnect sensors with little to no interruption in the time they have eyes on the patient, without having to turn attention away from the patient.

As with the medical device, display regions of a sensor hub display, in some embodiments, are user-selectable to trigger illumination on the corresponding cable. In this way, a user interacting with a sensor hub having multiple cables may swiftly identify correspondence between particular data and a particular cable.

Other capabilities and benefits of the teachings herein are described below.

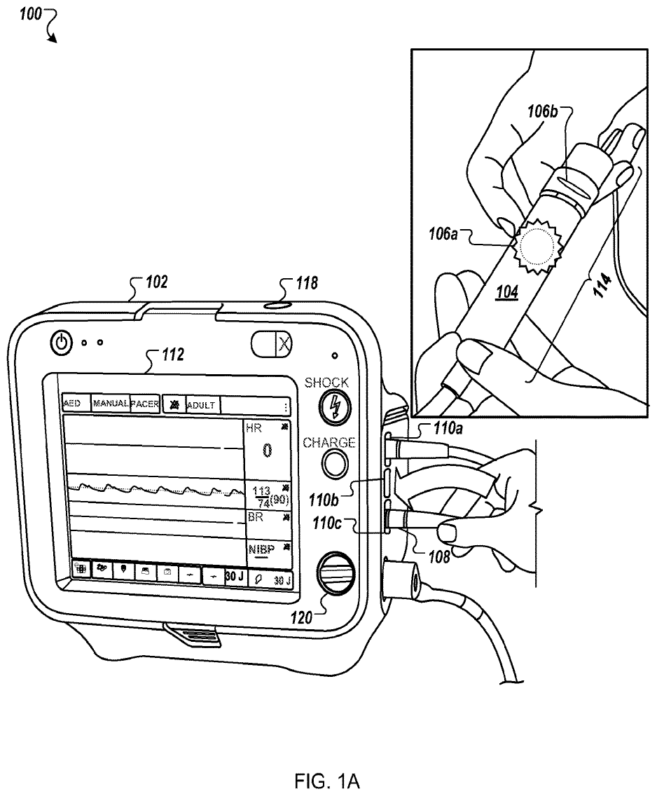

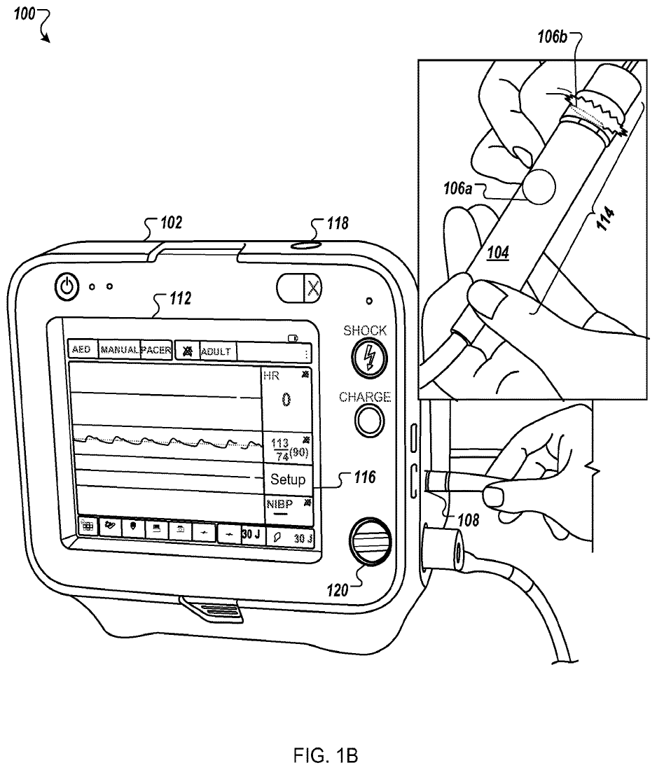

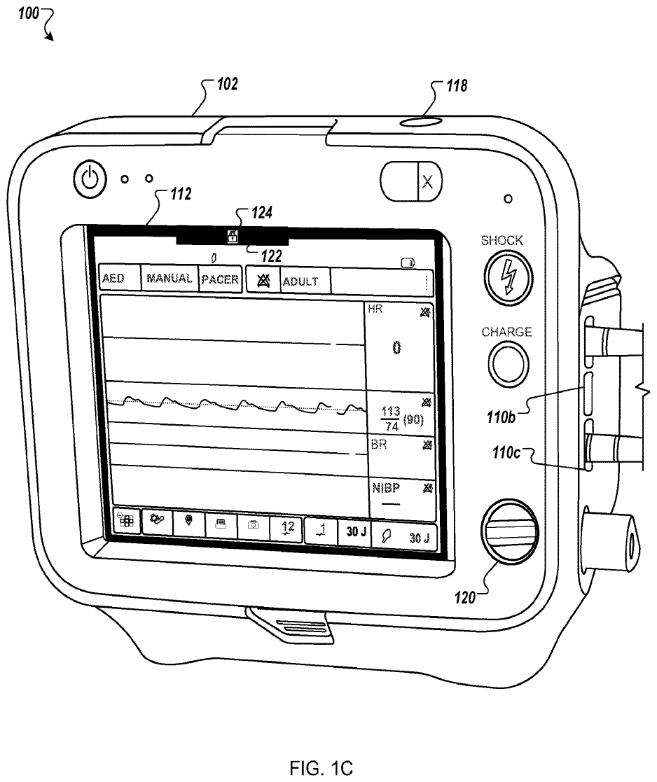

FIG. 1A and FIG. 1B illustrate cable state feedback mechanisms in an example system 100 including a medical device 102 configured with multiple sensor-agnostic data interface ports and a data transfer cable 104 connected thereto. The medical device 102, for example, may provide therapy to and/or monitor a patient and/or monitor treatment metrics for a treatment provided by a caregiver. As illustrated, the medical device 102 includes a display 112 presenting a user interface. The user interface may provide treatment data (e.g., medical, resuscitation, etc.) and/or physiological data gathered by various sensor devices connected to the medical device 102 via one of a set of data interface ports 110, such as a sensor device connected via the data transfer cable 104.

In some implementations, when a caregiver engages a medical device connector 108 of the data transfer cable 104 with one of the data interface ports 110 (e.g., port 110c, as illustrated), a first illumination element 106a (e.g., LED) disposed in a housing 114 of the data transfer cable 104 illuminates to acknowledge that the cable is connected to the medical device. For example, after detection of connection between the data transfer cable 104 and the data interface port 110c, a state of the data transfer cable 104 may transition from an unconnected state to a connected state, triggering illumination. The lighting of the first illumination element 106a, in some embodiments, is maintained for a duration that the data interface cable 104 is connected to the data interface port 110c of the medical device 102. In this manner, a caregiver can visually confirm that the sensor element or sensor device connected to the data transfer cable is engaged with the medical device 102 without needing to visit a location of the medical device 102.

In some implementations, after transitioning to the connected state, the medical device 102 and the data transfer cable 104 engage in an authentication routine. For example, the data transfer cable 104 may transition to an authentication/identification state. During authentication, the medical device 102 may confirm compatibility and authenticity of the data transfer cable 104, then enable data communications between the medical device 102 and the data transfer cable 104. Upon success of authentication, in some embodiments, one or more regions of a housing 114 of the data transfer cable 104 are illuminated to signify that the data transfer cable is now in communication with the medical device 102. In some examples, an indicator light may illuminate or move from a pending state (e.g., flashing, yellow, etc.) to an authenticated state (e.g., stable illumination, green, etc.). The medical device 102 may further enable powering of a sensor device connected to the data transfer cable 104. At this point, the state may be considered to be a port active state. Upon activating the sensor device and confirming intercommunication therewith, in some embodiments, one or more regions of the housing 114 of the data transfer cable 104 are illuminated to signify that the sensor device connected to the data transfer cable 104 is now issuing sensor signals captured by the medical device 102. In some examples, an indicator light may illuminate or move from a pending state (e.g., flashing, yellow, etc.) to a connected state (e.g., stable illumination, green, etc.).

Turning to FIG. 1B, a successful authentication of the data transfer cable 104, in some embodiments, results in illumination of a second illumination element 106b, alerting the caregiver that the sensor or sensor device attached to the data transfer cable 104 is connected to the medical device 102. The second illumination element 106b may remain illuminated while the state of the data transfer cable 104 remains in the port active state.

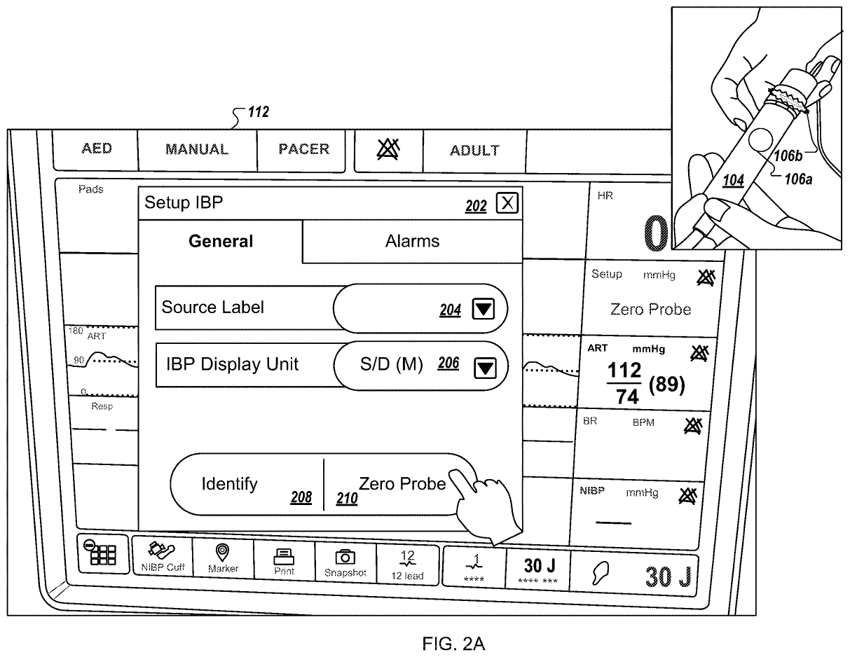

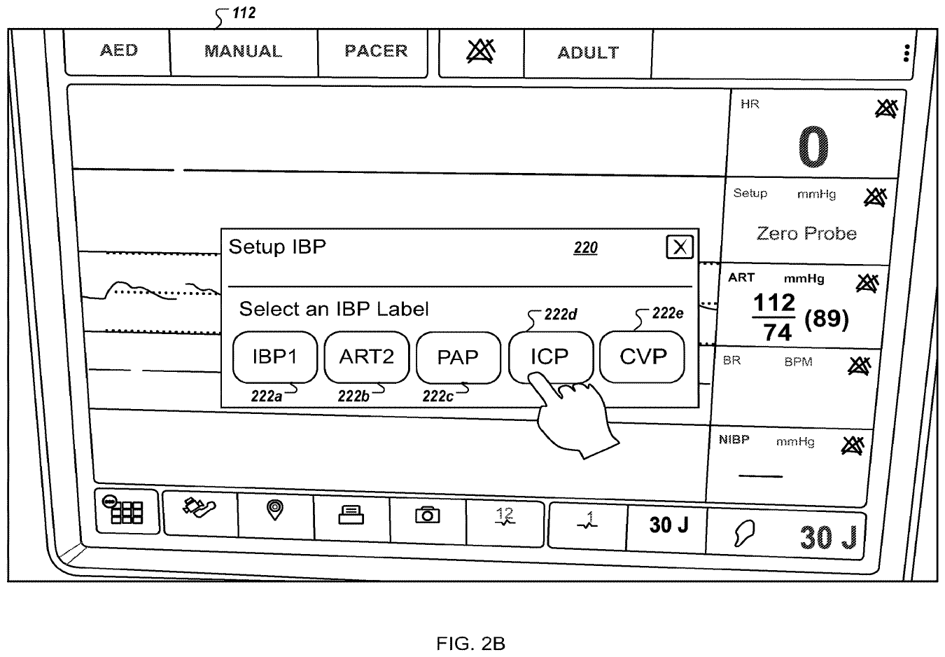

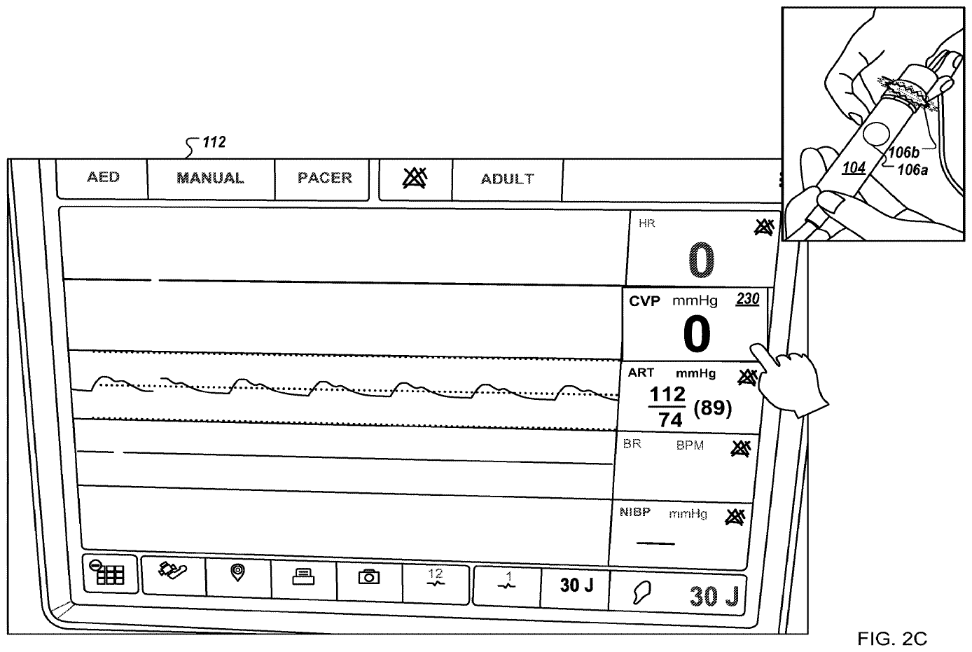

After transitioning to the port active state, in some implementations, additional setup is required prior to using certain sensors or sensor devices. As illustrated in FIG. 1B, for example, a setup message 116 is presented in a user interface region of the display 112 corresponding to the sensor attached to the data transfer cable 104. A user may select the region 116 of the setup message (e.g., using a touch gesture if the display 112 is touch-enabled, using a mechanical input device or mechanism, such as a navigation dial 120 on the front of the medical device 102, etc.), for example, to engage in a setup routine. In another example, turning to FIG. 2A through FIG. 2C, a series of stages of an invasive blood pressure (IBP) sensor setup routine are illustrated.

In some implementations, turning to FIG. 2A, the IBP sensor setup routine begins with presenting an interactive setup dialog in the user interface of the display 112 of the medical device 102. The interactive setup dialog, for example, is illustrated in a pop-up window 202 presented over a portion of the user interface of the display 112. The pop-up window 202, in some embodiments, is displayed upon selecting the region 116 including the setup message displayed in FIG. 1B (e.g., by use of a touch-sensitive display 112, through turning the navigation dial 120 and pushing the dial 120 inward to make a selection, etc.). In other embodiments, the pop-up window 202 is automatically launched upon successful transition into port active state and recognition, by the medical device 102, that the sensor attached to the data transfer cable 104 is an IBP sensor. The pop-up window 202 includes options for setting a source label 204 (e.g., IBP, ART, PAP, ICP, CVP, etc.) and selecting a display format 206 for metrics display (e.g., S/D (M), S/D, (M) S/D, (M), etc. where S=systolic, D=diastolic, and M=mean).

Further, the pop-up window 202 includes an identify control 208 for identifying the IBP probe corresponding to the pop-up window. Upon selecting the identify control 208, for example, the first illumination element 106a and/or the second illumination element 106b may be controlled to provide a visual confirmation to a caregiver of the data transfer cable 104 connected to the probe involved in the setup pop-up window 202. In some examples, one or both of the illumination elements 106a, 106b may change color and/or flash on and off for a period of time (e.g., up to a few seconds). In one example, upon initiation of setup (e.g., including the launch of the setup window 202), the second illumination element 106b may flash, change color, increase in intensity, or otherwise draw attention to the probe connected to the data transfer cable 104. If, further to the example, a caregiver failed to notice the visual identification (e.g., flashing, strobing, etc.) displayed by the second illumination element 106b, the caregiver may select the identify control to confirm which probe corresponds to the pop-up window 202.

Additionally, the pop-up window 202 includes a zero probe control 210 for zeroing (initializing) the IBP probe sensor. In some implementations, the second illumination element 106b is illuminated in a first color representing that a user action is needed (e.g., yellow, orange, red, etc.) and a second color (e.g., green, blue, white, etc.) representing that the data transfer cable 104 and/or the sensor device connected thereto are in a ready or active state.

Upon selection of the zero probe control 210, the first illumination element 106a and/or the second illumination element 106b may be controlled to provide a visual indication to a caregiver that the sensor of the IBP probe connected to the data transfer cable 104 is engaged in the zeroing process. As illustrated, for example, the second illumination element 106b is illuminated. In other examples, one or both of the illumination elements 106a, 106b may change color and/or flash on and off for a period of time (e.g., up to a few seconds).

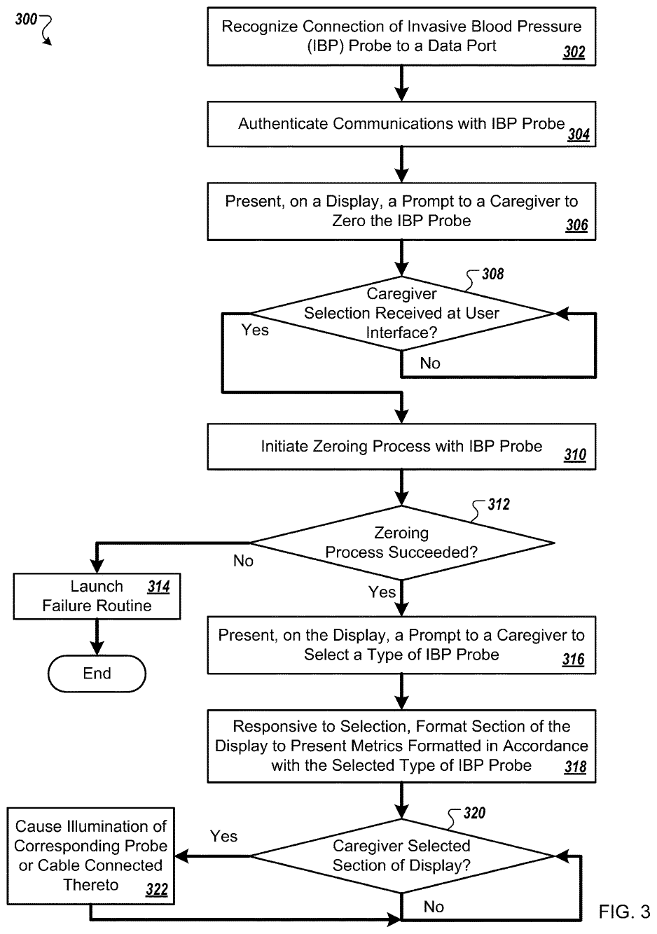

Turning to FIG. 3, a method 300 for automatically initializing an invasive blood pressure (IBP) probe connected to a sensor agnostic data interface port of a medical device is illustrated. The method 300, for example, may be performed by the medical device 102 and the data transfer cable 104 of FIG. 1A and FIG. 1B. The method 300, for example, may involve a series of user interactions via the display 112 of the medical device 102, as illustrated in relation to FIG. 2A through FIG. 2C.

In some implementations, the method 300 begins with recognizing connection of an invasive blood pressure (IBP) probe to a data port of a medical device (302). One or more connection detection cable contacts of the data port, for example, may be used by the medical device to detect connection/disconnection between a data transfer cable and a data port of the medical device. Coupling the data transfer cable to the data port, for example, may involve detecting a ground connection between the data port at the data transfer cable. Connection recognition, for example, may be performed in a manner described in relation to FIG. 1A.

In some implementations, communications with the IBP probe are authenticated (304). For example, the data transfer cable and the medical device may exchange authentication signals/messages during initiation of a communication coupling to verify that the data transfer cable and/or the IBP probe are recognized by and compatible with the medical device. The authentication, for example, may be performed as described in relation to FIG. 1A and FIG. 1B.

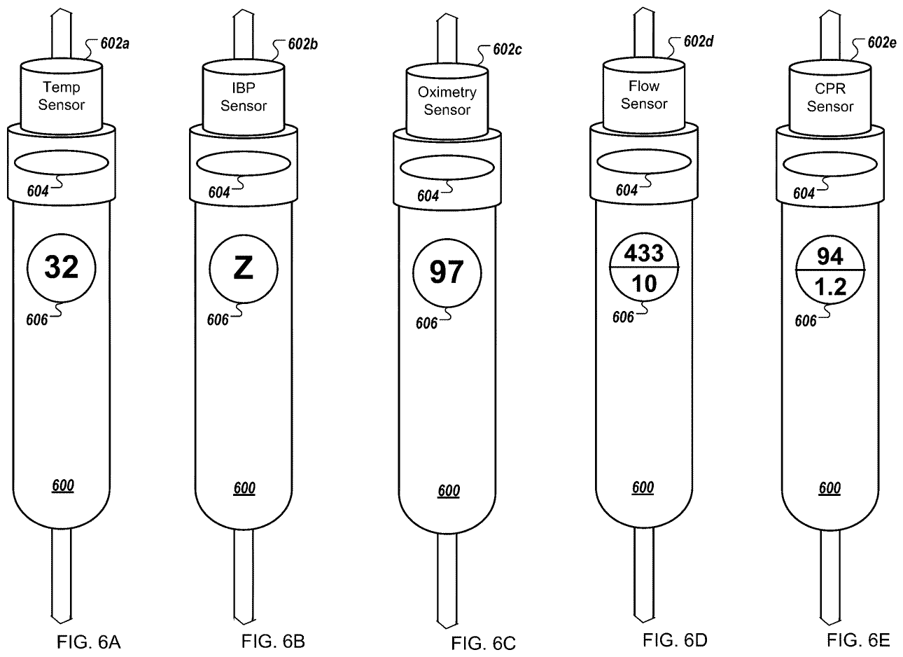

In some implementations, a prompt to a caregiver to zero the IBP probe is presented on a display (306). To ensure that pressure is measured accurately by an IBP probe, upon connection of a transducer of an IBP probe to a medical device, the transducer may need to be zeroed (e.g., the transducer may need to be vented to atmospheric air for a period of time). Thus, prior to use of an IBP probe, the zeroing process is performed on the IBP probe. For example, as shown in FIG. 2A, the pop-up window 202 presents the zero probe control 210 for caregiver selection. In another example, as shown in FIG. 6B, the IBP probe may include an alphanumeric display region such as an illumination element 606 presenting a zeroing prompt (illustrated in FIG. 6B as the letter “Z”).

In some implementations, the method pends until a caregiver selection is received at a user interface (308) responsive to the prompt. The selection, for example, may be made through a touch-sensitive display of the medical device, a mechanical interface of the medical device (e.g., the navigation dial 120 of FIG. 1A and FIG. 1B), or another input device in communication with the medical device. For example, the IBP probe may be rendered unusable until the zeroing process has been performed. In other implementations, opening of the stopcock to vent the transducer to the atmosphere may be detected by the medical device, thereby automatically initiating the zeroing process without need for the caregiver to provide an acknowledgement via the medical device.

In some implementations, a zeroing process with the IBP probe is initiated (310). For example, a caregiver may automatically open a stopcock to vent the transducer to the atmosphere. In another example, the probe may automatically vent itself responsive to a zeroing command.

In some implementations, if the zeroing process fails (312) a failure routine is launched (314). Failure, in some examples, can include a pulsation in the pressure channel, excessive noise in the signal, and/or a transducer offset greater than a predetermined threshold. Failure, for example, may be presented at the display of the medical device. The failure routine, for example, may include presenting, on the display, the same pop-up window 202 of FIG. 2A with the zero probe control 210 unselected so that the caregiver may repeat the zeroing process. In another example, a coaching message may be provided, such as a message instructing the user to “fully open the stopcock.”

If, instead, the zeroing process succeeds (312), in some implementations, a prompt is presented on the display to the caregiver to select a type (e.g., pressure source) of the IBP probe (316). Types of IBP probes, in some examples, can include abdominal aorta pressure (ABP), arterial pressure (ART), central venous pressure (CVP), intracranial pressure (ICP), pulmonary artery pressure (PAP), umbilical artery pressure (UAP), aorta (AO), brachial artery pressure (BAP), femoral artery pressure (FAP), labial artery pressure (LAP), radial artery pressure (RAP), and/or umbilical venous pressure (UVP). In some embodiments, the type of probe may be recognized by the data transfer cable 104 (e.g., based on information provided via the connected probe) and provided to the medical device 102 as a default label presented on the graphical user interface 112 (e.g., for optional override by the caregiver).

Turning to FIG. 2B, a pop-up window 220 overlaying a portion of the display 112 of the medical device 102 invites a caregiver to select an IBP label. Controls for a set of labels 222 are presented, including an IBP1 label 222a, an ART2 label 222b, a PAP label 222c, a CVP label 222d, and an ICP label 222e. As illustrated, the second control is marked “ART2” because the user interface 112 already includes a first ART display region 224 corresponding to another IBP probe connected to the medical device 102.

Returning to FIG. 3, in some implementations, responsive to selection, a section of the display is formatted to present metrics related to the IBP probe in accordance with the selection (318). The metrics, in some examples, can include systolic, diastolic, and mean values. A waveform, additionally, may be presented demonstrating changes in invasive pressure over time. The section of the display, for example, may be configured to present both the label (e.g., “IBP1” or “ART2” or “PAP” or “CVP” or “ICP”) as well as one or more metric values. As illustrated in FIG. 2C, for example, display region 224 is labeled “ART” and presents 112/74 (89 ). Further, a display region 230 corresponding to the newly zeroed probe is labeled “ICP” and presents a single value of 12.4 mmHg.

In some implementations, during operation, when a caregiver selects the section of the display corresponding to the IBP probe (318), the corresponding probe or cable connected thereto is illuminated (322). As described in relation to FIG. 1B, for example, the second illumination element 106b may light up, flash, change color, or otherwise draw attention to the probe corresponding to the second of the display (e.g., region 116) selected by a caregiver. Further, as illustrated in FIG. 2C, the second illumination element 106b may strobe or flash to draw attention to the probe corresponding to the display region 230.

Although the method 300 is described in accordance with a particular series of steps, in other embodiments, the method 300 includes more or fewer operations. For example, a display format corresponding to the display format control 206 of FIG. 2A may be initialized for the IBP probe. Additionally, in some embodiments, certain steps of the method 300 may be performed in a different order and/or in parallel. For example, prior to initiating the zeroing process with the probe (310), the caregiver may select the section of the display (320) to cause illumination (322), thereby confirming the caregiver is holding the appropriate probe for zeroing. Other embodiments of the method 300 are possible.

Turning to FIG. 1C, in some embodiments, a touch input functionality of the display 112 may be disabled, thereby allowing personnel to manipulate the medical device 102 and/or maneuver within close quarters of the medical device 102 without inadvertently entering commands or altering the contents of the display 112. In some examples, a manual lock control on the medical device 102 may be actuated to disable touch inputs or an input device in communication with the medical device 102 (e.g., a smart phone application, laptop application, remote control, wireless keyboard, etc.) may be used to disable touch inputs. As shown, a lock button 118 on the surface of the medical device 102 may be pressed. In another illustrative example, a lock slide button (not illustrated) may be shifted to a lock position, to disable touch inputs.

In some implementations, upon disabling the touch-sensitive interface of the medical device 102, a lock enabled indicator is presented within the display 112. For example, as illustrated, a lock indication frame 122 surrounds the graphical interface presented on the display 112, including a lock icon 124. The lock indication frame 122, for example, may be brightly colored (e.g., red, lime green, fuchsia, etc.) to draw attention to the touch screen having been disabled. In other examples, the lock enabled indicator may include a lock enabled message on the display 112 (e.g., in an upper or lower region of the display 112), a lighted indicator on the case of the medical device 102 (e.g., lighting up the lock button 118, etc.), and/or a message displayed upon a user contacting the surface of the display 112 (e.g., “touch interface unavailable” in a pop-up window or as a display overlay, etc.).

Upon disabling the touch interface feature of the medical device 102, in some implementations, a user may navigate the contents of the display 112, navigate a list of options (e.g., a drop-down menu), and/or alter parameter settings of the medical device 102 using a manual control on the medical device 102 and/or another device in communication with the medical device 102 (e.g., a smart phone application, laptop application, remote control, etc.). A navigation dial 120, illustrated in FIG. 1C, may be a rotary knob configured to be twisted clockwise to navigate to the right and counterclockwise to navigate to the left. Further, twisting clockwise and counter-clockwise may result in downward and upward movement within a list, such as a drop-down menu. In another illustrative example, a joystick style control may be provided on or in communication with the medical device 102 for submitting user interface interactions while the touch-sensitive display is disabled. While navigating, individual frames or options may be highlighted within the display 112 to illustrate a current position of focus. Further, a cursor icon may be presented on the display 112 to assist a user in navigating. In some embodiments, the feature of navigating options in the display 112 using a manual control such as the navigation dial 120 is disabled while the touch screen feature is activated.

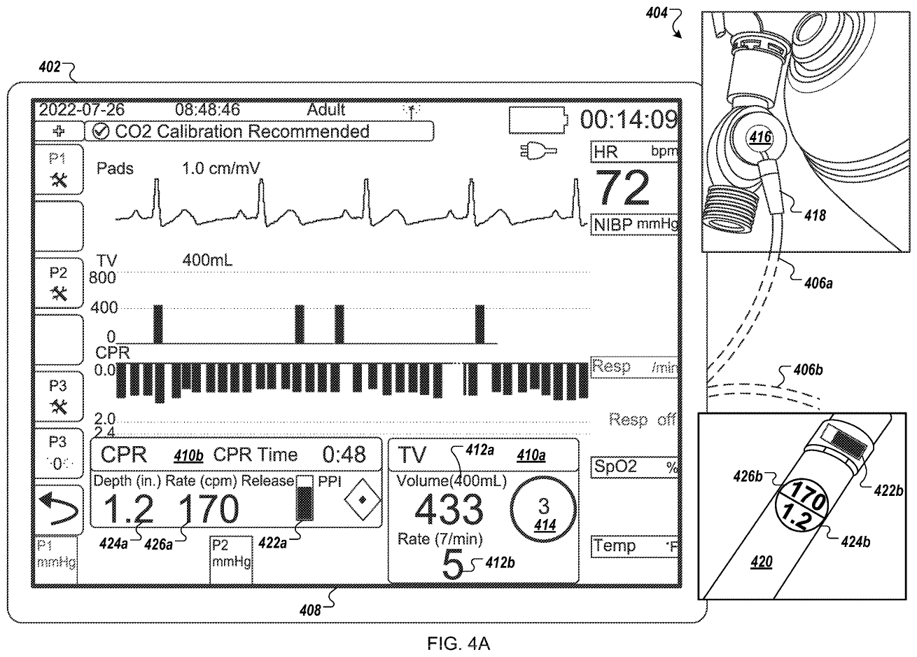

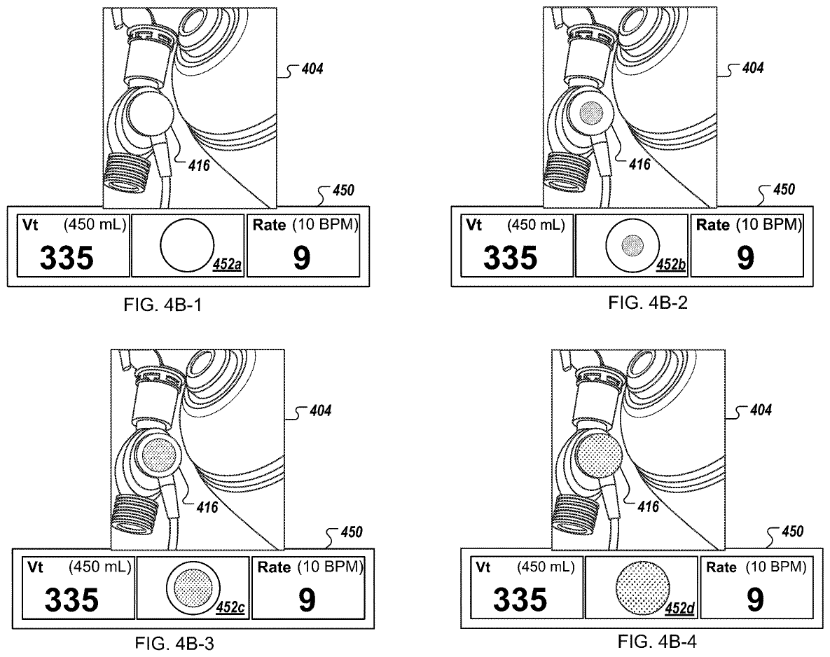

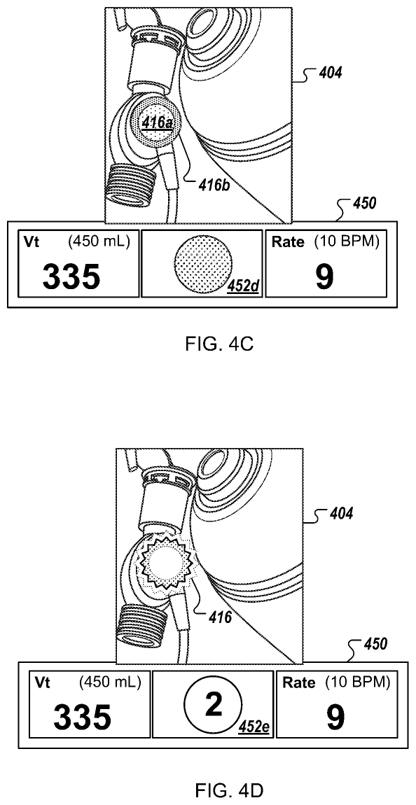

FIG. 4A and FIG. 4B-1 through FIG. 4B-4 illustrate example user interface output of a medical device 402 connected to a ventilation system 404 via a data transfer cable 406. Turning to FIG. 4A, a display region 408 of the medical device 402 is presenting both a ventilation feedback display region 410a and a compression feedback display region 410b. The compression feedback display region 410b, for example, may be generated based on a compression sensor device attached to the medical device 402 via a separate data transfer cable. The ventilation feedback display region 410a, for example, may be generated based on a ventilation sensor device attached to the medical device 402 via a separate data transfer cable, described further below.

In the ventilation feedback display region 410a, a volume 412a (e.g., 433 mL) and a rate 412b (e.g., 5 ventilations per minute) are represented. The volume 412a and rate 412b, for example, may be indicative of feedback 412 related to recent ventilation delivery. Further, the ventilation feedback display region 410a includes a countdown icon 414 indicative of coaching feedback presented to a caregiver for performing a next ventilation.

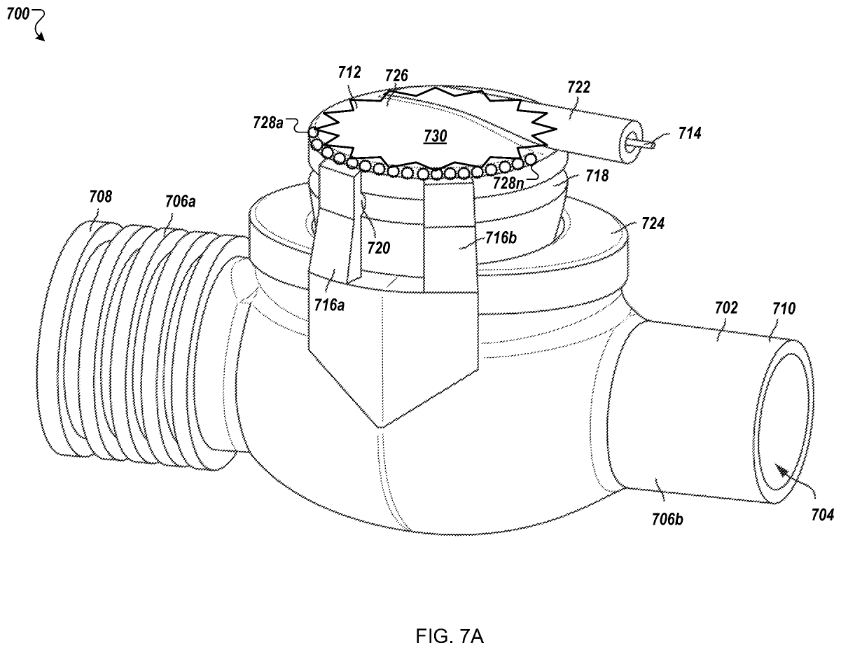

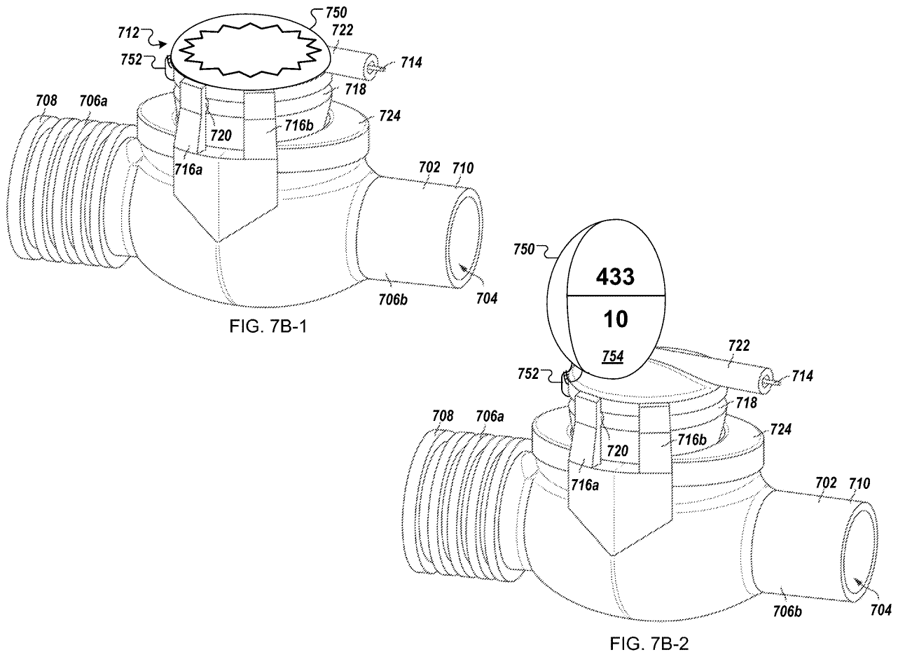

The ventilation system 404 includes an illumination element (e.g., LED display) 416. The illumination element 416 is disposed on or proximate to a connector 418 of the data transfer cable 406. In some embodiments, the illumination element 416 is part of the data transfer cable, described in greater detail in relation to FIG. 7A, below.

In some implementations, the illumination element 416 is controlled by the medical device 402 and/or a processor of the data transfer cable 406 to reproduce a portion of the feedback 412 and/or the coaching prompt 414. For example, turning to FIG. 4B-1 through FIG. 4B-4, the illumination element 416 of the ventilation system 404 is illustrated presenting a display coordinating with a ventilation feedback display region 450 for presentation on a medical device, such as the medical device 402 of FIG. 4A. In FIG. 4B-1, the illumination element 416 of the ventilation system 404 is blank (e.g., non-illuminated, illuminated in a basic color such as white, etc.), and a corresponding circle in a prompting graphic 452a of the ventilation feedback display region 450 is also blank or empty. The empty circle, for example, may prompt a caregiver to initiate a next ventilation using the ventilation system 404.

Turning to FIG. 4B-2, the illumination element 416 of the ventilation system 404 has an illuminated or colorful (e.g., green, yellow, etc.) smaller circle in a center of the illumination element 416 that extends for less than half the radius of the circular illumination element 416. A corresponding circle in a prompting graphic 452b of the ventilation feedback display region 450 is also filled in approximately the same manner (e.g., using a color, fill pattern etc.). The partially filled circle, for example, may prompt the caregiver to continue delivering air to a patient via the ventilation system 404 (e.g., a target volume has not yet been reached). For example, when the circle in the prompting graphic 452b of the ventilation feedback display region 450 is filled with the color yellow, indicating that the ventilation tidal volume and/or ventilation rate is outside of the respective target range(s), then the illumination element 416 of the ventilation system 404 may be illuminated with the same yellow color. Similarly, when the circle in the prompting graphic 452b of the ventilation feedback display region 450 is filled with the color green, indicating that the ventilation tidal volume and ventilation rate is within of the respective target ranges for both ventilation tidal volume and ventilation rate, then the illumination element 416 of the ventilation system 404 may be illuminated with the same green color. Accordingly, the yellow warning color may provide an indication that one or both of ventilation tidal volume and rate are outside of the desired target range such that the caregiver would need to consider how to adjust his/her application of manual ventilations for both parameters. Similarly, the green color may provide an indication that both ventilation tidal volume and rate are within the desired target range, giving the caregiver comfort that the manner in which he/she is providing manual ventilations is clinically desirable. The partially filled circle, for example, may prompt the caregiver to continue to deliver air to a patient via the ventilation system 404 (e.g., a target volume has not yet been reached), or simply may indicate that air is being delivered to the patient via the ventilation system 404.

FIG. 4B-3 shows that the illumination element 416 of the ventilation system 404 has an illuminated or colorful (e.g., green, yellow, etc.) larger circle in a center of the illumination element 416 that extends for greater than half the radius of the circular illumination element 416. A corresponding circle in a prompting graphic 452c of the ventilation feedback display region 450 is also filled in approximately the same manner (e.g., using a color, fill pattern etc.). The increasingly filled circle presented via both the illumination element 416 and the ventilation feedback display region 450, for example, may provide feedback to the caregiver that the air delivery to the patient is getting closer to the target volume.

In FIG. 4B-4, the circular illumination element 416 of the ventilation system 404 is substantially illuminated. A corresponding circle in a prompting graphic 452d of the ventilation feedback display region 450 is also completely filled (e.g., using a color, fill pattern etc.). The fully colored in/illuminated circle presented via both the illumination element 416 and the ventilation feedback display region 450, for example, may provide feedback to the caregiver that the air delivery to the patient has reached the target volume. At this point, the displays 416, 450 may remain essentially identical for a pause period of time prior to delivery of the next ventilation, in some embodiments, such that the caregiver will again be prompted to deliver air to the patient by the prompting display rendered in FIG. 4B-1. In some embodiments, the partial illuminated filling of the illumination element 416 shown in FIGS. 4B-2 and 4B-3 are optional, such as only the blank or complete illuminated filling of the illumination element 416 of FIGS. 4B-1 and 4B-4 are implemented, respectively, without partial illumination. In other implementations, full illumination is represented as shown in FIG. 4B-3, while an outer ring of illumination is used for providing additional information, such as quality/sufficiency feedback.

Turning to FIG. 4C, for example, an inner portion 416a of the circular illumination element 416 of the ventilation system 404 is substantially illuminated as in FIG. 4B-3, while an outer ring 416b of the circular illumination element 416 has been illuminated in a different manner (e.g., different color, illumination quality, etc.). The outer ring 416b, for example, may be illuminated in green to indicate that the volume and/or timing of air delivery was within target range, while the outer ring 416b may be illuminated in yellow to indicate that the volume and/or timing of the air delivery did not meet the desired target. The corresponding circle in the prompting graphic 452d of the ventilation feedback display region 450 is the same as presented in FIG. 4B-4.

In some implementations, a caregiver is prompted with a countdown or other differing display between ventilation deliveries. Turning to FIG. 4D, in some implementations, during a pause period of time prior to delivery of the next ventilation, the circular illumination element 416 of the ventilation system 404 is strobed or flashed. The strobing or flashing may be performed, for example, as a countdown to the next ventilation. A corresponding circle in a prompting graphic 452e of the ventilation feedback display region 450 presents a numeric value (e.g., number of seconds remaining until next ventilation).

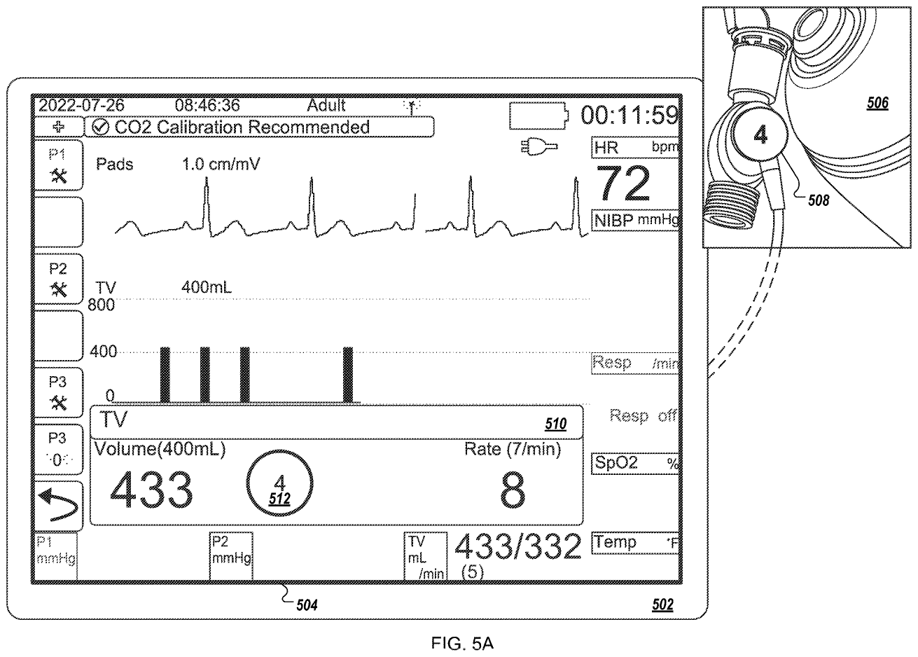

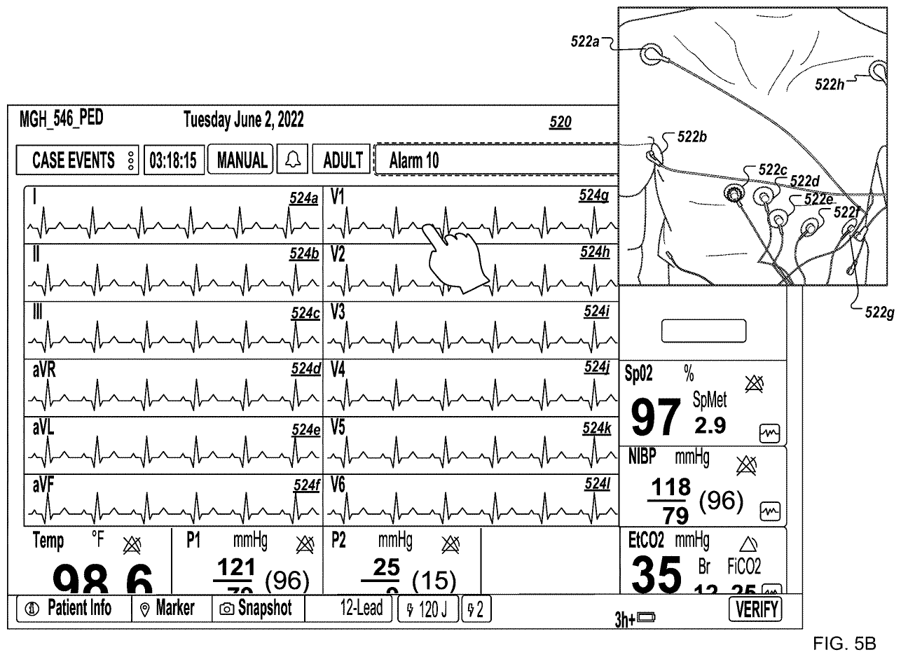

Turning to FIG. 5A, another example of coordination between a display region 504 of a medical device 502 and an illuminated display 508 of a ventilation system 506 is presented. As shown in FIG. 5A, a ventilation feedback section 510 of the display region 504 includes a circular prompting graphic 512 of the ventilation feedback section 510 including the number “4.” Correspondingly, a “4” is presented on the illuminated display 508 of the ventilation system 506. The number, for example, may be a countdown in seconds until next air delivery to the patient. In other embodiments, rather than or in addition to a numeric value, a set of countdown “tics”, “blocks” or a graduated bar may alter fill, appear, or disappear gradually during a pause period of time between ventilations.

FIG. 5B illustrates example user interface output coordinated between a display region 520 of a medical device and a set of ECG electrodes 522. The display region 520, for example, illustrates graphic outputs associated with a set of ECG channels 522a-1. The set of ECG electrodes 522, for example, may be connected to a medical device by a data transfer cable comprising electrical wires with electrode-receiving (e.g., “snap”) connectors connected to each ECG electrode 522. In some embodiments, the ECG sensor data corresponding to each individual ECG electrode 522 is logically linked to its respective output in the display region 520 such that, upon selection of a particular ECG channel 522, the ECG electrode(s) 522 corresponding to the ECG signals used to produce the output of the selected graph and/or the connectors leading thereto are illuminated (e.g., lit up, flashed, change color, etc.) to identify the source of the particular data display. For example, as illustrated in FIG. 5B, upon selecting ECG channel V1 522g, a connector portion of electrode 522c is illuminated. In this manner, if a particular graph appears to be producing inconsistent data, an error, or otherwise unexpected graphic output, a caregiver can quickly identify the corresponding ECG electrode(s) 522 and confirm positioning/adherence to the patient.

FIG. 6A though FIG. 6E illustrate a sensor-agnostic data interface (SA-DI) cable 600 including a first illumination element 604 (e.g., one or more LEDs for representing status) and a second illumination element 606 (e.g., an LED or LCD display). In each figure, the SA-DI cable 600 is coupled to a different type of sensor or sensor device 602, causing the feedback presented via the second illumination element 606 to change accordingly.

Turning to FIG. 6A, a temperature sensor element 602a is coupled to the SA-DI cable 600, and the second illumination element 606 is presenting a numeric value corresponding to a temperature (e.g., “32”) measured by the temperature sensor element 602a (e.g., temperature probe, etc.). The font (e.g., color, bold, flashing) of the numeric value and/or a fill within the region behind the font rendering of the numeric value, in some examples, may be indicative of whether the temperature is within an acceptable range or outside a threshold range of patient temperature.

In some implementations, a fill region behind the numeric value indicates a mode of therapy. For example, during patient cooling, the region behind the numeric value may be filled with a cool tone (e.g., blue), while, during patient warming, the region behind the numeric value may be filled with a warm tone (e.g., orange or pink).

Turning to FIG. 6B, an IBP sensor element 602b is coupled to the SA-DI cable 600, and the second illumination element 606 is presenting a “Z” corresponding to a zeroing process for initializing a transducer of the IBP probe connected to the SA-DI cable 600. In other embodiments, the second illumination element 606 may present “Zero” or other suitable prompting. The “Z” (or “Zero”, etc.) may be indicative of a prompt to a caregiver to initiate the zeroing process and/or the Z may be presented during the zeroing process. While the probe is in use, the illumination element 606 may present one or more numeric pressure measurements, such as systolic, diastolic, and/or mean pressure. The font (e.g., color, bold, flashing) of the numeric value and/or a fill within the region behind the font rendering of the numeric value, in some examples, may be indicative of whether the pressure is within an acceptable range or outside a threshold range of invasive blood pressure measurements.

Turning to FIG. 6C, a pulse oximetry sensor element 602c is coupled to the SA-DI cable 600, and the second illumination element 606 is presenting a numeric value corresponding to a blood oxygen saturation level (e.g., “97”) measured by the pulse oximetry sensor element 602c. The font (e.g., color, bold, flashing) of the numeric value and/or a fill within the region behind the font rendering of the numeric value, in some examples, may be indicative of whether the blood oxygen saturation level is within an acceptable range or outside a threshold range.

Turning to FIG. 6D, a flow sensor element 602d is coupled to the SA-DI cable 600, and the second illumination element 606 is presenting a set of numeric value corresponding to a volume (e.g., “433”) and rate (e.g., “10”) measured by the flow sensor element 602d. The set of numeric values are separated by a bar, such that each numeric value is disposed in a respective half circle of the illumination element 606. The font (e.g., color, bold, flashing) of the numeric value and/or a fill within the semi-circular region behind the font rendering of the numeric value, in some examples, may be indicative of sufficiency of the present metric (volume and/or rate). For example, both the upper and lower semi-circles may be filled with a same color/pattern (e.g., green, indicating sufficiency of both rate and volume) or a color/pattern of the upper half may be different than a color/pattern of the lower half (e.g., upper yellow, indicating a volume just outside of the sufficiency range for ventilation volume, and lower red, indicating a rate further outside of the sufficiency range for ventilation delivery).

In other embodiments (not illustrated), the upper half and the lower half of the display region may be reduced in size to present an outer ring encircling the upper half and lower half of the display region. The outer ring, for example, may represent a time to next ventilation, where a filled portion of the outer ring increases as time gets closer to the next ventilation time. The fill may include a gradient or single tone fill that gradually filles around the outer ring (e.g., clockwise or counterclockwise) to present a visual countdown until the next ventilation.

In further embodiments, the separator between the numeric regions of the illumination element 606 may be used as an indicator of a time to ventilation. For example, the bar may gradually change color and/or alter in thickness (increase or decrease) to represent a countdown to a next ventilation.

Turning to FIG. 6E, a CPR sensor element 602e is coupled to the SA-DI cable 600, and the second illumination element 606 is presenting a set of numeric value corresponding to a rate (e.g., “94”) and depth (e.g., “1.2”) measured by the CPR sensor element 602e. The set of numeric values are separated by a bar, such that each numeric value is disposed in a respective half circle of the illumination element 606. The font (e.g., color, bold, flashing) of the numeric value and/or a fill within the semi-circular region behind the font rendering of the numeric value, in some examples, may be indicative of sufficiency of the present metric (rate and/or depth).

Compression rates and/or compression depths, in some embodiments, are visually represented in at least two separate colors (e.g., by font and/or by fill) based on ranges of rates and/or depths. For example, compression rate may be designated as “slow”, “sufficient,” or “fast,” (e.g., orange, green, yellow) and color markings or other visual indication may be adjusted appropriately. In another example, compression rate may be designated as “outside sufficient range” or “inside sufficient range” (e.g., green for within the target range, yellow or red for outside the target range). In a further example, compression rate may be designated as “far outside range”, “dropping outside range”, or “sufficient”, with either three (e.g., red, yellow, green) options for indication or five options for indication (e.g., to differentiate visibly between too slow or too fast).

In other embodiments, a bar graph or half-pie graph corresponding to each numeric value may be filled in correspondence to sufficiency in relation to a current metric. For example, a partially filled graph may represent slower than sufficient, a filled graph may represent sufficient, and a filled region (e.g., with red) may represent faster than a maximum target rate. Similarly, turning to compression depth, overcompression/undercompression may be dealt with in a similar manner or in two different manners (e.g., sufficient compression or not sufficient compression; undercompression, sufficient compression, or overcompression, etc.). Visual indications may be applied in a similar fashion as described in relation to compression rate.

FIG. 7A and FIG. 7B illustrate a flow sensor system including an illuminated user interface for providing coaching and/or feedback to a caregiver supplying ventilation to a patient. The flow sensor systems, for example, may be used in the ventilation systems of FIG. 4A, FIG. 4B-1 through FIG. 4B-4, and/or FIG. 5A.

Turning to FIG. 7A, in some implementations, a flow sensor system 700 includes a flow conduit 702 that defines a lumen 704 allowing for the passage of gas through the flow conduit 702 from a ventilation source, such as a manual ventilation bag or automated ventilation system, to a patient.

In some embodiments, the flow conduit 702 is made up of a first piece 706a and a second piece 706b molded from a thermoplastic material, which are assembled and ultrasonically welded to form the flow conduit 702. The first piece 706a may include a first connection portion 708, and the second piece 706b may include a second connection portion 710 to which the flow sensor may be connected. For example, the first connection portion 708 may be configured to couple to a ribbed fitting (e.g., for a ventilator tube or ventilation port), and the second connection portion 710 may be configured to couple with a tapered fitting that slides along the surface thereof (e.g., over or under the surface of the conduit) to form a friction or interference type fit. It can be appreciated that the connection portions 708, 710 may have any appropriate configuration for establishing a suitable port connection with a tube or conduit.

In some implementations, the flow sensor system 700 is configured to be placed in communication with a ventilation assembly for delivering gas through the lumen 704 of the flow conduit 702. The ventilation assembly may include a manual bag ventilation system and/or may comprise an automated ventilation system. As such, the first connection portion 708 on the first piece 706a may be configured with an inner diameter having a larger diameter suitable for receiving an end of a tube or passageway of the ventilation assembly to form a standard female connection. The first connection portion 708 may also have a ribbed outer diameter to form a standard male connection with a larger tube into which the first connection portion 708 is inserted. The second connection portion 710 on the second piece 706b may have a smooth outer diameter to form a standard male connection within another tube or passageway of the ventilation assembly.

The flow sensor system 700, in some implementations, is configured to be connected to a processor of a computing device (e.g., a patient monitor) and/or a processor of a data transfer cable by a cable connector 714. The processor, for example, may analyze sensor signals from the flow sensor system 700, derive metrics, and provide the operator of the flow sensor system 700 with feedback concerning the flow rate and/or volume of the gas being delivered to the patient. A portion of the feedback, for example, may be presented on a housing of a data transfer cable coupled to the cable connector 714 of the flow sensor system. Further, a portion of the feedback may be presented on a display of the computing system (e.g., patient monitor). The feedback may be visual, audio, and/or haptic feedback. This feedback may allow the operator to adjust the timing and/or force of the actuations of the manual bag ventilation system.

In some implementations, the flow sensor system 700 includes a cover 724 over a circuit board configured to collect sensor data related to the flow sensor system 700. The cover 724, as illustrated, is positioned on the upper surface of the flow conduit 702. In some embodiments, both the circuit board and the cover 724 are shaped to correspond to the shape of the upper surface of the flow conduit 702 to fit on the flow conduit 702 and hold the circuit board in place.

In some implementations, a processor is incorporated in a connector 712 of the flow sensor system 700, which is configured to be removably coupled to the flow conduit 702. Upon coupling, for example, the connector 712 may place the circuit board and/or pressure sensors of the flow sensor system 700 in communication with the processor of the connector 712. That is, when the connector 712 is coupled to the flow conduit 702, electrical communication may be established between the pressure sensors, the circuit board, the processor, and other electrical components (e.g., computing device, defibrillator, tablet, monitor, etc.) to which the cable 714 extends. The outer housing of the connector 712 may include a strain relief housing 722 that surrounds the cable 714 as it passes through the housing in order to protect the connection between the cable 714 and the processor.

In some implementations, an illuminated display region 726 of the connector 712 is configured to present at least a portion of the feedback concerning the flow rate and/or volume of the gas being delivered to the patient. This feedback may allow the operator to adjust the timing and/or force of the actuations of the manual bag ventilation system. The display region 726, as illustrated, includes a series of illumination elements 728 surrounding an edge of the illuminated display region 726 (e.g., elements including element 728a and 728n and spanning in between, as illustrated). The display region 726 may further include an illuminating display 730 on a surface of the connector 712 (e.g., LCD display, LED display, etc.).

In some implementations, the illuminated display region 726 is configured provide visual feedback to the caregiver as to whether or not the ET tube has been properly placed. When the tube is determined to be properly placed, the system may activate a green LED at a suitable location (e.g., along the edge of the connector 712 using at least a portion of the illumination elements 728a-728n, on a surface of the connector 712 using the illuminating display 730, etc.). If the previous ventilation attempt resulted in the determination of an improperly placed ET tube, then the system may activate a red LED of the visual indicator. The visual indicator may also include a series of LEDs configured as a dual color bar-graph to indicate the tidal volume of each successive ventilation, with the color of the LED bars indicative of whether or not the tube is properly placed (green indicating proper placement; red-indicating improper placement). Alternatively, separate indicating lights may be provided for airway and breathing, to indicate proper ET tube placement and ventilation tidal volume, respectively.

In some implementations, the connector 712 includes a molded outer shell or housing that contains the internal assembly of the connector 712, such as the processor. When the connector 712 is mounted on the flow conduit 702, the housing may be engaged by snap arms 716a, 716b extending from the upper surface of the flow conduit 702. The housing may include an indentation 718 formed therein and each of the snap arms 716a, 716b may include a protuberance 720 formed along its length. The protuberances 720 on the snap arms 716a, 716b may engage within the indentation 718 on the housing to maintain the engagement between the connector 712 and the snap arms 716a, 716b while allowing the connector 712 to rotate with respect to the flow conduit 702 without becoming disengaged and allowing the connector 712 to be coupled to the flow conduit 702 from a variety of angular orientations. The snap arms 716a, 716b may be flexible so that they may deflect a suitable amount to allow the connector 712 to be connected to and removed from the flow conduit 702. It can be appreciated that the complementary snap arms 716a, 716b and protuberances 720 are not required aspects of the present disclosure, as the flow conduit 702 and the connector 712 may be engaged via any suitable configuration, such as via magnetic coupling, interference fit, amongst others.

In some embodiments, when connected to the flow conduit 702, the connector 712 may swivel about a transverse axis of the flow conduit 702, similar to that of a turret. In the configuration shown in FIG. 7A and FIG. 7B, the cable 714 extends horizontally relative to the flow conduit 702 along the plane about which the connector 712 swivels. Thus, the feedback presented to the caregiver via the illuminated display 726, if including numeric feedback, may lack appropriate orientation with respect to the caregiver.

Turning to FIG. 7B-1 and FIG. 7B-2, in some implementations, the connector 712 includes a domed display attachment 750 including one or more illumination elements. The domed display attachment 750, for example, may provide feedback that is visible from a number of orientations (e.g., above or from any side of the domed display attachment 750). The domed display attachment 750, in some embodiments, is built into the connector and electrically connected to the cable 714. In other embodiments, the domed display attachment 750 is attached to the connector 712 via a display attachment 752. The display attachment 752, for example, may deliver power and control signals from the cable 714 via the connector 712.

Turning to FIG. 7B-2, in some implementations, the display attachment 752 is a hinged attachment configured to enable the domed display attachment 750 to move into a vertical position, thereby exposing a display surface 754. As illustrated, the display surface 754 is presenting a volume (433) as well as a rate (10) in numeric format. The display surface 754, for example, may be an LED or LCD display. In some embodiments, the display attachment 752 is rotatably attached to the connector 712, such that the face of the display surface 754 may be oriented to be directed toward a caregiver. In some embodiments, the caregiver may rotate the connector 712 to direct the display surface 754 into view.

Returning to FIG. 7A, in other embodiments (not shown), the cable 714 may extend vertically relative to the flow conduit 702. For example, instead of swiveling about a two-dimensional plane with a circular range of motion, the connector 712 and flow conduit 702 engagement may be constructed such that the cable 714 may have a generally hemispherical or dome-like range of motion.

In some implementations, the flow sensor system 700 includes a connector 712 that places first and second absolute pressure sensors in electronic communication with the processor. The processor may be configured receive the absolute pressure measurements from the first and second absolute pressure sensors and may determine at least one of a flow rate and a volume of gas flowing through the lumen 704 of the flow conduit 702 based on the pressure measurements in the flow conduit 702. The processor may also be configured to generate a signal outputting the determined flow rate and/or volume of gas flowing through the flow conduit 702 and/or to send a feedback signal to adjust the gas flow through the lumen 704 of the flow conduit 702 based on at least one of the determined flow rate and the volume of gas flowing through the lumen 704. In other implementations, the processor may simply store/transmit signals arising from the pressure sensors to another computing device for further analysis and processing, such as the medical device the data transfer cable is connected to. In further embodiments, the processor of the flow sensor system 700 may perform some of these calculations, such as determining the flow rate through the conduit 702 based on the signals sent from the pressure sensor (e.g., based on a pre-calibrated pressure look up table) and may further perform a mathematical integration resulting in the flow volume. An external device (e.g., tablet, defibrillator, patient monitoring device, etc.) may then receive those values of rate and volume and send feedback signals to the processor, which may be further output via the illumination display in an intuitive manner for guiding or otherwise encouraging a user to maintain and/or improve the overall quality of resuscitation.

It can be appreciated that each of the processes for analyzing the measurement signals produced by the pressure sensor(s), outputting a calculated value (e.g., flow rate, flow volume, peak inspiratory pressure (PIP), etc.), and providing a feedback signal to adjust the manner in which gas flow is provided based on the analysis of the pressure measurement signals may be performed at any suitable part(s) and location(s) of the overall medical system. For example, each of these steps may be performed at the same location by the same processor, such as a processor located in the cable head or connector of the data transfer cable. Alternatively, each of these steps may be performed at different locations of the medical system by different processors (e.g., located in the cable head connector, another part of the flow sensor, a more distantly located medical device system such as a defibrillator, monitor, tablet, computer, ventilator, etc.). For instance, the processor located in the cable head connector may analyze pressure measurement signals and determine the flow rate and/or volume of flow within the lumen 704 of the flow sensor system 700. As noted above, this same processor, or another processor (e.g., located in a monitor, defibrillator, ventilator, amongst other suitable processing systems), may further output a feedback signal to adjust flow parameters (e.g., ventilation bagging, automated ventilation characteristics).

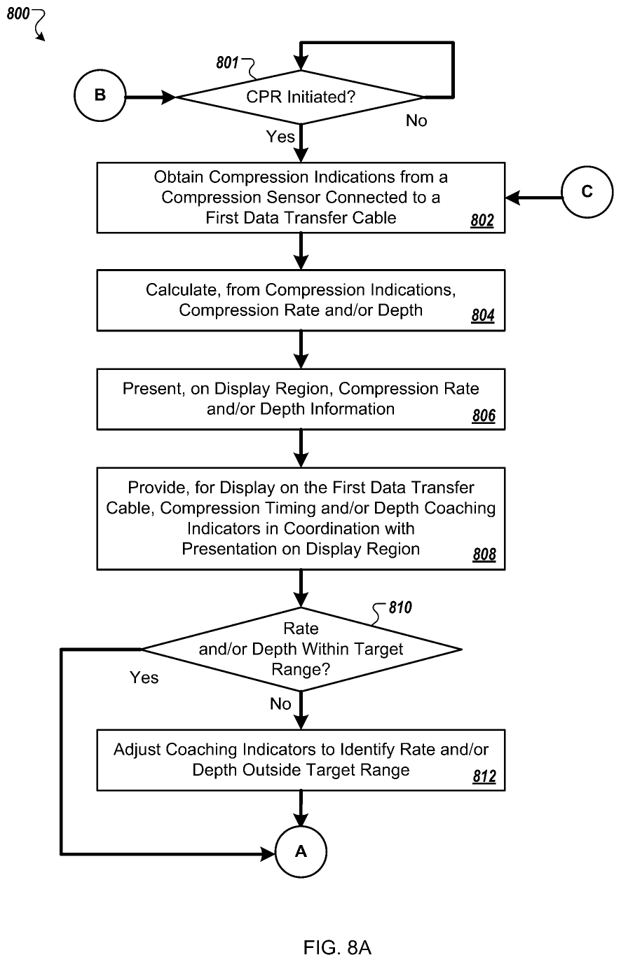

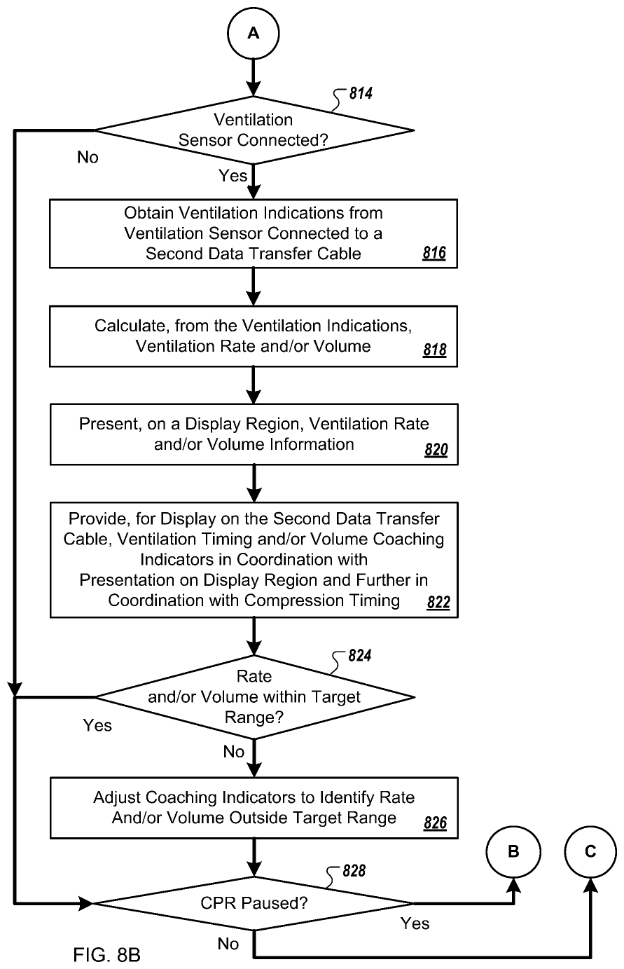

FIG. 8A and FIG. 8B illustrate a flow chart of an example method 800 for coordinating caregiver feedback between a computing device having a display and one or more sensor devices connected to a medical device by one or more data transfer cables. The data transfer cable(s) may include SS-DI cables and/or SA-DI cables, as described above. The method 800, for example, may be performed by the medical device 102 of FIG. 1A and FIG. 1B, the medical device 402 of FIG. 4A, and/or the medical device 502 of FIG. 5A. In some embodiments, portions of the method 800 are performed by a computing device in communication with a medical device, such as a tablet computer or laptop computer.

In some implementations, the method 800 begins with determining that CPR has been initiated (801). For example, a medical device such as a patient monitoring and/or treatment device may obtain initial sensor data from a compression sensor, such as the compression sensor 602e of FIG. 6E. In another example, a user may submit, at a control device, an indication of CPR initiation. The control device, in some examples, may include a portable patient monitoring device, a tablet computing device, or a defibrillation device.

In some implementations, compression indications are obtained from a compression sensor connected to a first data transfer cable (802). The compression indications, for example, may be provided to a medical device from the compression sensor via the first data transfer cable. Further, the medical device may provide the compression indications to a separate computing device, such as a tablet computing device.

In some implementations, compression rate and/or compression depth are calculated from the compression indications (804). In some embodiments, the compression sensor data is aggregated, adjusted, or at least partially converted to compression metrics by a processor of the first data transfer cable prior to being provided to the medical device. The compression sensor data, in further embodiments, is aggregated, adjusted, or at least partially converted to compression metrics by a processor of the medical device prior to being provided to the computing device. The compression rate and/or compression depth may be calculated, at least in part, by a processor of the first data transfer cable, a processor of the medical device, and/or the processor of a computer device, in various implementations.

In some implementations, compression timing and/or depth information are presented on a display region (806). The display region may be a display region of the medical device and/or the computing device. The compression timing and/or depth information, for example, may be presented in a manner as illustrated in FIG. 4A, including a depth in inches (e.g., 1.2) and/or a rate in compressions per minute (cpm) (e.g., 170).

In some implementations, compression timing and/or depth coaching indicators are provided for display on the data transfer cable in coordination with presentation on a display region (808). For example, as illustrated in FIG. 4A, a CPR release timing bar graph 422a is mirrored on a first illumination element of a data transfer cable 420 as a bar graph 422b. Further, metrics feedback related to a depth of compressions 424a (e.g., 1.2 inches) and a rate of compressions 426a (e.g., 170 compressions per minute), as presented on the display 408, are mirrored on a second display element of the data transfer cable 420 as a rate of compressions 426b and a depth of compressions 424b.

In some implementations, if the calculated rate and/or depth of compressions is not within a target range (810), the coaching indicators are adjusted to identify that the rate and/or depth is outside of target range (812). For example, a blinking illumination, warning color of LED indicators (e.g., red or yellow), warning symbol in an LCD display, or other indicator may be provided to convey to the caregiver that the therapy delivery is outside of target ranges.

Turning to FIG. 8B, in some implementations, if a ventilation sensor is connected via a second data transfer cable (814), ventilation indications are obtained from the ventilation sensor (816). The ventilation indications, for example, may be provided to a medical device from the ventilation (e.g., air flow) sensor via the second data transfer cable. Further, the medical device may provide the ventilation indications to a separate computing device, such as a tablet computing device.

In some implementations, a ventilation rate and/or a ventilation volume is calculated from the ventilation indications (818). In some embodiments, the ventilation sensor data is aggregated, adjusted, or at least partially converted to ventilation metrics by a processor of the second data transfer cable prior to being provided to the medical device. The ventilation sensor data, in further embodiments, is aggregated, adjusted, or at least partially converted to ventilation metrics by a processor of the medical device prior to being provided to the computing device. The ventilation rate and/or ventilation volume may be calculated, at least in part, by a processor of the second data transfer cable, a processor of the medical device, and/or the processor of a computer device, in various implementations.

In some implementations, the ventilation rate and/or the ventilation volume is presented on a display region (820). The display region may be a display region of the medical device and/or the computing device. The ventilation rate and/or the ventilation volume information, for example, may be presented in a manner as illustrated in FIG. 4A, including a volume in milliliters (e.g., 433) and/or a rate in ventilations per minute (e.g., 5).

In some implementations, ventilation timing and/or volume coaching indicators are provided for display on the second data transfer cable in coordination with display in the display region and further in coordination with the compression timing (822). For example, as illustrated in FIG. 5A, the ventilation feedback section 510 of the display region 504 includes the circular prompting graphic 512 of the ventilation feedback section 510 including the number “4” (e.g., 4 seconds until the next ventilation). Correspondingly, a “4” is presented on the illuminated display 508 of the ventilation system 506. Similarly, FIG. 4B-1 through FIG. 4B-4 illustrates an example of display feedback 452 mirroring feedback presented on the illumination element 416 of the ventilation system 404.

In some implementations, if the calculated rate and/or the calculated volume of ventilation is not within a target range (824), the coaching indicators are adjusted to identify that the rate and/or the volume is outside of target range (826). For example, a blinking illumination, warning color of LED indicators (e.g., red or yellow), warning symbol in an LCD display, or other indicator may be provided to convey to the caregiver that the therapy delivery is outside of target ranges.

In some implementations, if CPR is paused (828), the method 800 returns to pending initiation of CPR (802). Otherwise, the method 800 returns to obtaining compression indications (802).

Although the method 800 is described in accordance with a particular series of steps, in other embodiments, the method 800 includes more or fewer operations. For example, prior to providing for display the ventilation timing coaching indicator (822), the timing may be calculated based in part on tracking of number of compressions. Additionally, in some embodiments, certain steps of the method 800 may be performed in a different order and/or in parallel. For example, although described as a series of operations, obtaining the compression indications (802) and obtaining the ventilation indications (816) may occur substantially in real-time and in parallel by the first data transfer cable and the second data transfer cable, respectively. Further, calculating the compression indications (804) and presenting the compression rate and/or depth information (806) may occur substantially in parallel and in real time along with the calculating the ventilation rate and/or volume (820) and the presenting the ventilation rate and/or volume information (820). Other embodiments of the method 800 are possible.

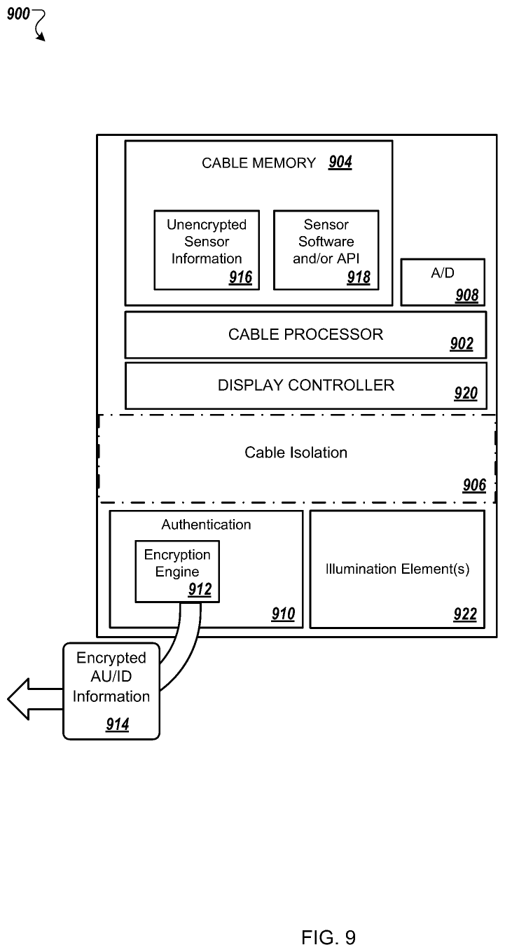

FIG. 9 illustrates a schematic diagram of example cable circuitry 900 for a data transfer cable. The cable circuitry 900, for example, may be included in a housing portion of a data transfer cable, such as the housing 114 of the data transfer cable 104 of FIG. 1A and FIG. 1B or the housing of connector 712 of FIG. 7A and FIG. 7B. The cable circuitry 900 includes a cable processor 902, a cable memory 904, and a cable patient leakage current isolation device and/or circuitry 906. In some implementations, the cable circuitry 900 includes an analog-to-digital (A/D) converter 908 configured to convert analog signals from at least one sensor in communication with the circuitry 900 to digital signals for the cable processor 902. The A/D converter 908 is shown separately from the cable processor 902 for clarity but may be integrated into the cable processor 902.

The cable patient leakage current isolation device and/or circuitry 906, in some implementations, includes circuitry and other hardware and/or physical components configured to limit patient leakage current flow from an attached medical device such as the medical device 102 of FIG. 1A and FIG. 1B, the medical device 402 of FIG. 4A, and/or the medical device 502 of FIG. 5A, to the patient via a sensor in communication with the data transfer cable. Particularly for high voltage electrotherapy, leakage current isolation can be beneficial for safety reasons. In an illustrative example, the medical device may be a defibrillator and the patient may be coupled to defibrillation electrodes as well as to a sensor, in turn, is coupled to the medical device via a data transfer cable including the cable circuitry 900. Further to the example, defibrillation current (I_{defibrillation}) follows a current path from the medical device to the defibrillation electrode, through the patient to the electrode, and then back to the medical device. There is a potential current path between this defibrillation circuit and the data transfer cable, for example, via stray capacitance between the medical device, the patient and the sensor, by which a patient leakage current (I_{leakage}) may reach the patient via the sensor. However, the cable patient leakage current isolation 906 in the cable circuitry 900 may be configured to prevent any patient leakage current from reaching the sensor and the patient, thus providing a protective layer of safety built into the data transfer cable.

The cable patient leakage current isolation 906, in some implementations, includes an isolation barrier device, for example a double capacitive isolation barrier device, a digital isolator device, an optical isolator device, etc. The cable patient leakage current isolation 906 may be configured to transmit power signals and communication signals across an isolation barrier. The hardware and/or physical components of the cable circuitry 900 may further include, in some examples, conductive and insulative layers and/or coatings coupled to and/or surrounding the cable patient leakage current isolation 906.

In some implementations, the cable patient leakage current isolation 906 is configured to transmit power unidirectionally across an isolation barrier toward the cable processor 902. When the data transfer cable is coupled to the medical device, the medical device may be the sensor power source and may provide power to the cable processor 902 and the sensor via the port of the medical device and the data transfer cable connected thereto. For example, the data transfer cable may transmit power via at least one conductive wire with another wire at ground. The cable patient leakage current isolation 906 may transfer this power, transmitted by the data transfer cable from the medical device, across the isolation barrier in one direction to the cable processor 902 and the sensor. With this unidirectional power transfer, there is substantially limited or no transmission of power from the processor side of the cable patient leakage current isolation 906 toward the medical device. In an example, the cable patient leakage current isolation 906 may transfer, or transmit, 0.1-1 Watts of power across the isolation barrier.

In some embodiments, the cable patient leakage current isolation 906 is configured to transmit an amount of power across the isolation barrier that is specific to the power requirements of the sensor connected to the data transfer cable. For example, an invasive blood pressure sensor may require approximately 0.2 Watts whereas a flow sensor may require approximately 0.5 Watts. Thus, the power transmission capability of the cable patient leakage current isolation 906 may be tailored to the power requirement of the sensor. As a result, the medical device may be configured to apply power in an amount compatible with a variety of sensors that may be connected to one of its data ports.

In some implementations, the cable patient leakage current isolation 906 is configured to transmit communication signals bi-directionally across the isolation barrier. The bi-directional nature of this transmission, for example, may enable the medical device to be a source of communication signals and send information via these signals to the cable processor 902 and a sensor connected to the cable. Similarly, this bidirectionality may enable the cable processor 902 and/or the connected sensor to be a source of communication signals and send information via these signals to the medical device.

In some implementations, the cable circuitry 900 includes authentication circuitry 910 for authenticating the data transmission cable with a medical device. The data transmission cable, for example, may include at least one authentication cable contact configured to connect with an authentication contact of a data port of the medical device, and the data cable's conductive wires may include at least one authentication wire. The authentication circuitry 910 may be configured to receive an authentication/identification (AU/ID) request from the medical device via the at least one authentication cable contact. Additionally, the authentication circuitry 910 may be configured to send AU/ID information back to the medical device in response to the received AU/ID request.

As shown in FIG. 9, in some implementations, the authentication circuitry 910 includes an integrated encryption engine 912 configured to use an encryption mechanism (e.g., encryption key(s), encryption algorithm(s), etc.) specific to the medical device. For example, a manufacturer of both the medical device and the data transfer cable may provide for encryption keys compatible with and unique to both the medical device and the data transfer cable for use in authenticating the data transfer cable. The encryption engine 912 may provide encrypted AU/ID information 914 to the medical device for use in authentication of the data transfer cable.

In some implementations, the cable memory 904 includes stored unencrypted sensor information 916. In the absence of a malicious and/or hacked modification of the data transfer cable, the unencrypted sensor information 916 matches the encrypted AU/ID information 914. The cable memory 904 may further include stored sensor software and/or application programming interface (API) 918 and corresponding software/API information such as, in some examples, a software version number, an API version number, update information, supported data protocols, and/or sensor data formats.

In various implementations, the sensor software and/or API 918 is stored in the memory 904 at the time of manufacture of the data transfer cable. The medical device, in some embodiments, is configured to provide any available updates this software when the data transfer cable is connected to the medical device. In some embodiments, sensor data formats are transmitted from the data transfer cable to the medical device, and data formats are updated on the medical device when the data transfer cable is connected to the medical device.

During communications with the data transfer cable, the medical device may receive version and update information for the sensor data format, software and/or API 918. In such embodiments, if an update is required, the medical device may instruct the cable processor 902 to enter a download mode. The cable processor 902, in response, can accept or reject this request based on other ongoing activities. Upon acceptance, the medical device may initiate and proceed with a sensor data format, software and/or API update to the software and/or API 918.

In some implementations, the cable circuitry 900 includes a display controller 920 for controlling one or more illumination elements 922. The display controller 920, in some examples, may cause illumination of different colors, fill patterns, strobing or blinking patterns, and/or intensities of illumination responsive to instructions from the cable processor 902. The different colors, fill patterns, strobing or blinking patterns, and/or intensities of illumination may be presented to convey different types of status indicators, warnings, caregiver feedback, and/or alarm states. Further, in some embodiments, the illumination element(s) 922 are configured to present alphanumeric characters, such as an LED display or LCD display. In these embodiments, in some examples, status indicators, warnings, caregiver feedback, and/or alarm states may include alphanumeric messages, such as messages presented in relation to the various sensors 602 on the illumination element 606 in FIG. 6A through FIG. 6E.

In various implementations, the medical device may be a defibrillator, patient monitor, defibrillator/monitor, an automated compression device, a therapeutic cooling device, an extracorporeal membrane oxygenation (ECMO) device, a ventilation device, combinations thereof, or another type of medical device configured to couple to one or more therapy delivery components to provide therapy to the patient. In an implementation, the medical device may be an integrated therapy delivery/monitoring device within a single housing. The single housing may surround, at least in part, the therapy delivery components and the monitoring components. In an implementation, the medical device may be a modular therapy delivery/monitoring device, with patient therapy components in one unit communicatively coupled to a patient monitoring unit without therapy delivery components.

The medical device may be, for example, a therapeutic medical device capable of delivering a medical therapy. For example, the medical therapy may be electrical therapy (e.g., defibrillation, cardiac pacing, synchronized cardioversion, diaphragmatic or phrenic nerve stimulation) and the medical device may be a defibrillator, a defibrillator/monitor, a mechanical ventilator such as the ZOLL Z-Vent, and/or another medical device configured to provide electrotherapy. As another example, the medical therapy may be chest compression therapy for treatment of cardiac arrest and the medical device may be a mechanical chest compression device such as a belt-based chest compression device or a piston-based chest compression device. As other examples, the medical therapy may be ventilation therapy, therapeutic cooling or other temperature management, invasive hemodynamic support therapy (e.g., Extracorporeal Membrane Oxygenation (ECMO)), etc. and the medical device may be a device configured to provide a respective therapy. In an implementation, the medical device may be a combination of one or more of these examples. The therapeutic medical device may include patient monitoring capabilities via one or more sensors. These types of medical therapy and devices are examples only and not limiting of the disclosure.

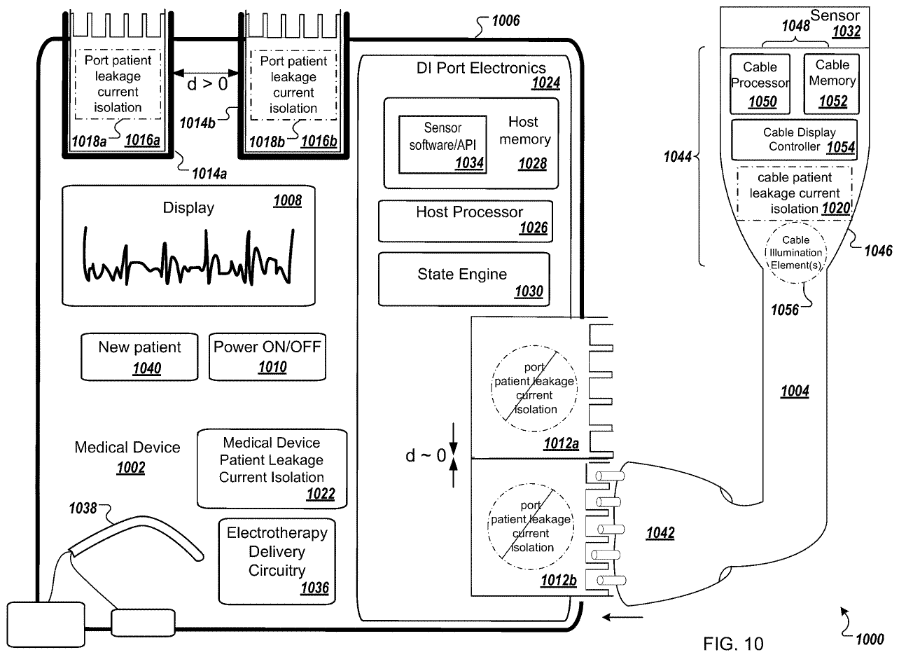

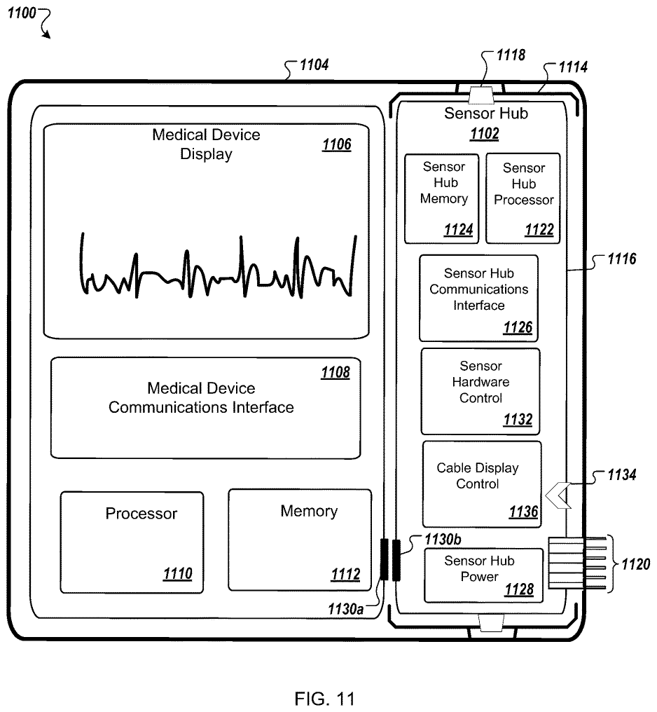

Turning to FIG. 10, a schematic diagram of an example medical device/data transfer cable system 1000 with sensor-agnostic data interface ports is shown. The medical device/data transfer cable system 1000 includes a medical device 1002 and the data transfer cable 1004. The medical device 1002 includes a housing 1006, a display 1008, a power control 1010, a new patient control 1040 for indicating operation of the system 1000 with a new patient, and at least one data transfer (e.g., sensor-agnostic data interface (SA-DI)) port 1012 (e.g., SA-DI ports 1012a and 1012b). Although two ports are shown in FIG. 10, this quantity of ports is an example only and not limiting of the disclosure. In various implementations, the medical device 1002 may include one or more SA-DI ports only or a combination of one or more SA-DI ports and one or more sensor-specific DI (SS-DI) ports, such as SS-DI ports 1016a and 1016b.

As illustrated schematically in FIG. 10, the SS-DI ports 1016a and 1016b each include port patient leakage current isolation 1018a, 1018b. The data transfer ports 1016a and 1016b are also spaced apart by a distance d>0 for noise reduction and electrical isolation. The data transfer ports 1016a and 1016b may also include a physical element 1014a, 1014b, such as, for example, a layer of conductive material, to reduce electromagnetic interference causing signal noise. As discussed above, these features of the SS-DI ports increase the weight and volume of the medical device 1002. In contrast, the SA-DI ports 1012a and 1012b may exclude (i.e., not include) port patient leakage current isolation. The SA-DI ports 1012a, 1012b are configured to couple to a data transfer cable compatible with the SA-DI ports 1012a, 1012b (e.g., with an electromechanical connector 1042 of the data transfer cable 1004). The compatible data transfer cable, as illustrated in cable 1004, includes cable patient leakage current isolation 1018. Additionally, the SA-DI ports 1012a and 1012b may be disposed proximate to one another at a spacing approximately equal to zero. Because these ports do not include patient leakage current isolation 1018, they do not require a noise reduction barrier or the physical layer 1014. The lack of a port patient leakage current isolation, inter-port spacing, and physical noise reduction layer in aggregate over multiple SA-DI ports reduces the weight and volume of the medical device 1002. For example, the medical device 1002 may accommodate more sensors with the SA-DI ports than with SS-DI ports and still realize an overall weight and volume reduction. Note that the medical device 1002 may include additional patient leakage current isolation 1022 beyond that provided by the data transfer cable 1004.

In some implementations, the medical device 1002 includes DI port electronics 1024. One or more components of the DI port electronics 1024 may be physically separate or separable from the medical device electronics (e.g., processor, memory, and associated electronics and hardware controls for therapy delivery, data collection, processing, analysis, communication, and display, etc.). One or more components of the DI port electronics 1024 may be communicatively and/or electronically coupled to the medical device electronics. In some embodiments, the DI port electronics 1024 are integrated into and/or components of the medical device electronics. The DI port electronics 1024 may include a host processor 1026, a host memory 1028, and a state engine 1030. In some embodiments, the state engine 1030 is a part of and/or a function of the host processor 1026. The host processor 1026 may receive sensor data from a sensor 1032 via the data transfer cable 1004 and may provide the sensor data to the medical device electronics for processing and/or display (e.g., at the physical display 1008). In some embodiments, the host memory 1028 includes stored sensor software and/or API 1034 and corresponding software/API information such as, in some examples, a software version number, an API version number, update information, and/or supported data protocols.

In some implementations, the medical device 1002 is configured as a defibrillator or a patient monitor/defibrillator. In such a configuration, the medical device 1002 may include electrotherapy delivery circuitry 1036 and defibrillation electrodes 1038 which may also serve as ECG sensors. For example, the electrotherapy delivery circuitry 1036 may include one or more capacitors configured to store electrical energy for a pacing pulse or a defibrillating pulse. The electrotherapy delivery circuitry 1036 may further include resistors, additional capacitors, relays and/or switches, electrical bridges such as an H-bridge (e.g., including insulated gate bipolar transistors or IGBTs), voltage measuring components, and/or current measuring components.

The display 1008, in some embodiments, is configured to provide at least one visual representation of sensor data received by the medical device 1002 via the SA-DI ports 1012a and/or 1012b and/or via the SS-DI port(s) 1016a, 1016b. The visual representations may provide the data as graphical and/or textual data. The visual representations may include waveform data, for example, but not limited to ECG, pulse oximetry, and/or capnography. The visual representations may include discrete numerical data, for example, but not limited to blood pressure (NIBP, IBP) heart rate, an instantaneous pulse oximetry value and/or an instantaneous capnography value. Additionally or alternatively, the visual representations may include or provide caregiver feedback, for example, cardiopulmonary resuscitation (CPR) feedback and/or ventilation feedback. The CPR feedback may include, for example, compression depth, compression rate, compression time, compression release, and/or perfusion performance. This display 1008 may provide the CPR feedback in real-time on a compression-by-compression basis. The ventilation feedback may include, for example, gas volume, ventilation rate, ventilation quality, and/or ventilation time. In an implementation, the ventilation feedback may be bag valve mask feedback. The visual representations may further include image data, for example, but not limited to, laryngoscopy and/or ultrasound images. The ultrasound images may include ultrasound images of a patient's tendons, muscles, joints, internal organs, skeletal structures, abdomen and/or the patient's heart, blood vessels, carotid artery, and/or other components of the cardiovascular system. The visual representations may be part of a guided medical intervention such as biopsies, tissue or fluid samples, and/or other diagnostic or invasive procedures.

The DI port electronics 1024 may control and handle data from the SA-DI ports 1012a, 1012b. The DI port electronics 1024 may further control and handle data from the SS-DI ports 1016a, 1016b. Alternatively, the SS-DI ports 1016a, 1016b may not connect electrically and/or communicatively to the DI port electronics 1024. For example, the medical device electronics may control and handle data from the SS-DI ports 1016a, 1016b.

In some implementations, the DI port electronics 1024 include the state engine 1030. The DI port state engine 1030, the DI port electronics 1024, and/or the host processor 1026 may manage each SA-DI port 1012a, 1012b independently from one or more other DI ports 1012a, 1012b. The DI port state engine 1030 may manage a state of the SA-DI ports 1012a, 1012b.

In some implementations, the data transfer cable 1004 compatible with the SA-DI ports 1012a, 1012b includes a flexible cable having conductive wires disposed within a continuous insulative sheath. The conductive wires may include single strands and/or multi strands of one or more conductive materials. The cable 1004 may be fixedly fastened to a first electromechanical connector 1044 at a first end of the cable 1004 and to a second electromechanical connector 1042 at a second end of the cable 1004.

In some implementations, the first electromechanical connector 1044 includes a housing 1046 and an electrical mating 1048 (e.g., a first electrical mating) disposed within the housing 1046 at an open end of the housing distal from the cable. In other words, the cable connects to the housing 1046 at a first end of the housing 1046 and the electrical mating 1048 for the sensor 1032 is disposed in a second and different end of the housing 1046. The electrical mating 1048 may be configured to detachably couple to the sensor 1032 (e.g., to an electrical connector associated with the sensor 1032). The electrical mating 1048, for example, may provide electrical coupling between one or more contacts associated with the sensor 1032 and one or more contacts associated with the data transfer cable 1004. The mating attachment of the electrical mating 1048 and the electrical connector 1044 may include, in some examples, a pin/socket combination, a plug/jack combination, or a card edge/spring contact combination.

The first electromechanical connector 1044, in some implementations, includes data interface circuitry disposed within the housing 1046. The data interface circuitry may be electrically coupled to the electrical mating 1048 by one or more electrical contacts and may be electrically coupled to the conductive wires of the cable 1004. The data interface circuitry may include a cable processor 1050, a cable memory 1052, and the cable patient leakage current isolation 1020. In some embodiments, the data interface circuitry includes an analog-to-digital (A/D) converter circuit configured to convert analog signals from the sensor 1032 to digital signals for the cable processor 1050. The A/D converter may be integrated into the cable processor 1050.

In some implementations, the cable patient leakage current isolation 1020 includes an isolation device and/or circuitry and other hardware and/or physical components configured to limit patient leakage current flow from the medical device 1002 to the patient via the sensor 1032. Particularly for high voltage electrotherapy, leakage current isolation can be beneficial for safety reasons. The cable patient leakage current isolation 1020 may include an isolation barrier device, for example a double capacitive isolation barrier device, a digital isolator device, an optical isolator device, etc. The cable patient leakage current isolation 1020 may be configured to transmit power signals and communication signals across an isolation barrier.

In some implementations, the cable patient leakage current isolation 1020 is configured to transmit power unidirectionally across an isolation barrier toward the cable processor 1050. When the data transfer cable 1004 is coupled to the medical device 1002 via the SA-DI port 1012a or 1012b, the medical device 1002 may be the sensor power source for the sensor 1032 and may provide power to the cable processor 1050.

In some implementations, the data transfer cable 1004 includes a cable display controller 1054, such as the display controller 920 of FIG. 9, configured to control illumination of one or more cable illumination elements 1056. The illumination elements 1056, as described herein, can be positioned in various locations on the cable, a cable housing, and/or connectors to the sensor 1032 and/or the data interface port 1012a, b and/or 1018a, b. The cable display controller 1054 and/or cable processor 1050, in some embodiments, is configured to present one or more sensor-agnostic status indicators such as, in some examples, a cable connected status indicator, a cable authentication status indicator, and/or a sensor connected status indicator. Further, the cable display controller 1054 and/or cable processor 1050. In some embodiments, is configured to present one or more sensor-specific status indicators such as, in some examples, a sensor reading outside target range status, caregiver feedback indicator(s), and/or a caregiver coaching/prompting indicator(s). Certain status indicators may be controlled by the medical device 1002 (e.g., by the host processor 1026). For example, upon selection of a portion of the display 1008 dedicated to the data transfer cable 1004, an identify status may be presented via the cable illumination element(s) 1056.

Turning to FIG. 11, a schematic diagram illustrates an example medical device 1100 having a removable sensor hub 1102. The removable sensor hub 1102 may be coupled to an inside or an outside of a medical device housing 1104. The medical device housing 1104 of the medical device 1100, in some embodiments, includes a medical device display 1106 (e.g., a first display), a communications interface 1108 (e.g., a first communications interface), a processor 1110 (e.g., a first processor), a memory 1112 (e.g., a first memory), and associated circuitry. The processor 1110 and the memory 1112 may be communicatively coupled to the display 1106 and the communications interface 1108.

In some implementations, the housing 1104 includes a sensor hub connector 1114. In various implementations, the sensor hub connector 1114 may be disposed within the housing 1104 (e.g., as illustrated) or may be disposed on the outside of the housing 1104 (e.g., on an exterior of the housing 1104). The sensor hub connector(s) 1114 disposed within the housing 1104 may, for example, form a receptacle configured to accept of the sensor hub 1102 and retain the sensor hub 1102 within the housing 1104. The receptacle may also be configured to release the sensor hub 1102 for removal from the medical device 1100. The sensor hub connector(s) 1114 disposed on the exterior of the medical device 1100 may include one or more of a bracket, a clip, a clamp, a magnet, a receptacle, etc. configured to secure the sensor hub 1102 to the exterior of the housing 1104. The sensor hub 1102 may include one or more mating mechanisms configured to removably couple to the sensor hub connector 1114. In an illustrative example, the mating mechanism may be a contour on the sensor hub 1102.

In some implementations, the sensor hub connector 1114 may enable a wired electrical and/or communicative coupling between the sensor hub 1102 and the medical device 1100 via one or more contacts. The sensor hub contacts 1130b (e.g., one or more first electrical contacts) may be disposed on the sensor hub 1102 and the medical device contacts 1130a (e.g., one or more second electrical contacts) may be disposed on the medical device 1100. The sensor hub connector 1114 may retain the sensor hub 1102 in a position in which these contacts touch one another.

In some embodiments, the sensor hub 1102 and the medical device 1100 communicate with one another via a wired connection when the sensor hub 1102 and medical device 1100 are physically coupled and/or decoupled. The sensor hub 1102, in some embodiments, is configured to communicatively couple via a network connection with one or more remote computing devices. The remote computing devices may include a server and other devices communicatively coupled via the server through the network such as a personal computer, laptop, tablet, mobile device, and/or other medical devices. In this manner, the sensor hub 1102 may enable telemedicine and remote data viewing, analysis, storage, and/or sharing.

The sensor hub 1102 includes a housing 1116 (e.g., a second housing or a sensor hub housing). The housing 1116 may include one or more mating mechanisms 1118 configured to removably couple the sensor hub 1102 to the sensor hub connector 1114. The sensor hub 1102, in some implementations, includes at least one DI port 1120 coupled to the housing 1116. The at least one DI port 1120 may include an SS-DI port (e.g., SS-DI ports 1016a 1016b of FIG. 10) and/or an SA-DI port (e.g., SA-DI port 1012a, 1012b of FIG. 10). The at least one DI port 1120 may be configured to couple to a data transfer cable (e.g., data transfer cable 1004 of FIG. 10) and a sensor (e.g., sensor 1032 of FIG. 10) and configured to receive sensor data.

The sensor hub 1102, in some implementations, includes a sensor hub processor 1122 (e.g., a second processor), a sensor hub memory 1124 (e.g., a second memory), and a sensor hub communications interface 1126 (e.g., a second communications interface). The at least one DI port 1120 may be communicatively coupled to the sensor hub processor 1122. The sensor hub processor 1122 may be configured to receive sensor data via the at least one DI port 1120 and send the sensor data to the sensor hub communications interface 1126.

In some implementations, the sensor hub processor 1122 is configured to store the sensor data in the sensor hub memory 1124. The sensor hub 1102 may include at least one universal serial bus (USB) port 1134. The sensor hub 1102 may include a sensor hub power source 1128. For example, the sensor hub power source 1128 may include one or more batteries configured to provide power to the sensor hub 1102 independently from power provided to the sensor hub 1102 by the medical device 1100. In an illustrative example, the one or more batteries may recharge using power from the medical device 1100 when the sensor hub 1102 is physically connected to the medical device 1100 with the sensor hub connector 1114 (e.g., via the electrical contacts 1130a, 1130b).

In some implementations, the sensor hub communications interface 1126 is configured to communicatively couple to the medical device communications interface 1108 and send the sensor data to the medical device processor 1110 via the medical device communications interface 1108. The medical device processor 1110, in some embodiments, is configured to control the medical device display 1106 to display a visual representation (e.g., a first visual representation) of the sensor data obtained via the sensor hub 1102 to a user.

In some implementations, the sensor hub communications interface 1126 and the medical device communications interface 1108 are configured to communicate with each other via wired and/or wireless communicative couplings. The communications interfaces 1108 and 1126 may communicate via a wired coupling (e.g., via the contacts 1130a and 1130b) when the sensor hub 1102 is physically retained by the sensor hub connector 1114. The communications interfaces 1108 and 1126 may communicate via a wireless coupling when the sensor hub 1102 is decoupled from the sensor hub connector 1114. The sensor hub connector 1114 and the mating mechanism 1118 may be configured to physically couple the medical device 1100 and the sensor hub 1102 such that the contacts 1130a and 1130b provide electrical and/or communicative connectivity.

The medical device 1100, in some implementations, includes multiple sensor hubs (e.g., multiple sensor hub connectors internal and/or external to the housing 1104). Each sensor hub may include a sensor hardware control 1132. The sensor hardware control 1132 may be tailored to a specific type of sensor or set of types of sensors. For example, each sensor hub of two or more sensor hubs may include sensor hardware control tailored to one or more specific types of sensors configured to couple to the particular sensor hub via the DI ports, such as DI port 1120. The quantity of DI ports and the types of sensors associated with the DI ports may vary from sensor hub to sensor hub. The processor 1110 of the medical device 1100 may be configured to provide visual representations of data from all of the one or more sensor hubs coupled to the medical device 1100 via the display 1106 of the medical device 1100.

In an illustrative example, the sensor hub 1102 may include the sensor hardware control 1132 for side stream capnography. Further, this sensor hub 1102 may be configured with two DI ports (e.g., such as DI port 1120)—a first DI port configured to connect to an SpO2 sensor and a second DI port configured to connect to a capnography sensor. The DI ports may be SA-DI ports with software/API appropriate for sensor data formats received from both SpO2 sensors and capnography sensors. The DI port 1120, in another example, may connect to an NIBP sensor and the sensor hardware control 1132 may include a pneumatic pump system for NIBP. These modular sensor hubs may be independently serviceable and replaceable and may enable the manufacturer and the customer to tailor the medical device 1100 to sensor capabilities and combinations according to specific customer needs.

In some implementations, the processor 1110 and/or the sensor hardware control 1132 includes and/or commands a cable display control 1136 for controlling output to display elements of a connected data transfer cable, such as the cable illumination element(s) 1056 of FIG. 10. The cable display control 1136 may be tailored to a specific type of sensor or set of types of sensors (e.g., a sensor integrated into or connected to an SS-DI data transfer cable). For example, each sensor hub of two or more sensor hubs may include cable display control 1136 tailored to one or more specific types of sensors configured to couple to the particular sensor hub via the DI ports, such as DI port 1120. In other embodiments, the cable display control 1136 may be agnostic to sensor type (e.g., for sensors designed to be interchangeably connected to an SA-DI data transfer cable).

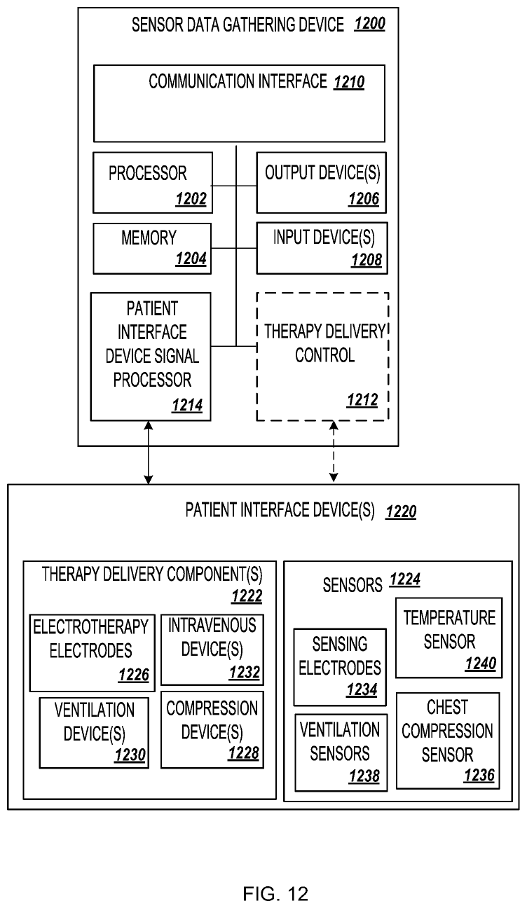

Turning to FIG. 12, examples of components of a sensor data gathering device 1200 (e.g., the medical device 1002 of FIG. 10, the sensor hub 1102 of FIG. 11, etc.) are shown schematically. The sensor data gathering device 1200 may include at least one processor 1202, at least one memory 1204, one or more output devices 1206, one or more user input devices 1208, and at least one communication interface 1210.

The communication interface 1210, in some implementations, is configured to transmit and/or receive information to and/or from one or more devices external to and communicatively coupled to the sensor data gathering device 1200. The communication interface 1210 may transmit and/or receive the information via a wired and/or wireless communicative coupling. The information may include information stored in the memory 1204. The information may include, for example, but not limited to, resuscitative treatment information, physiological information, patient information, rescuer and/or caregiver information, location information, rescue and/or medical treatment center information, etc.

The communication interface 1210, in some implementations, is configured to enable short-range and/or long-range wireless communication capabilities which may include communication via near field communication, ZigBee®, Wi-Fi, Bluetooth®, satellite(s), radio waves, a computer network (e.g., the Internet), a cellular network, etc. The communication interface 1210 may enable communication via a network such a Local Area Network (LAN), Wide Area Network (WAN), a mesh network, an ad hoc network, or another network. The communication interface 1210 may include, for example, an RS-232 port for use with a modem-based dialup connection, a copper or fiber 10/100/1000 Ethernet port, or a Bluetooth® or Wi-Fi interface.

In some implementations, the communication interface 1210 enables communication with one or more other computing or medical devices.

The output device(s) 1206 and user input device(s) 1208 may be included in the device 1200 and/or coupled to the device 1200. The output device(s) 1206 may include one or more of a display, a speaker, and a haptic device. The display may be a display screen. The medical device may provide at least one first display screen and the sensor hub may provide at least one second display screen. The display may provide a graphical user interface (GUI). The display may be, for example, but not limited to, a liquid crystal display (LCD) and/or a light emitting diode (LED) display.

In some implementations, one or more output device(s) 1206 are input/output device(s) capable of capturing user input. For example, a display device may be a touchscreen. The touchscreen may be, for example, a pressure sensitive touchscreen or a capacitive touchscreen. The touchscreen may capture user input provided via touchscreen gestures and/or provided via exertions of pressure on a particular area of the screen. Examples of touchscreen gestures that may enable user input may include pushing on the touchscreen to exert pressure that exceeds a particular threshold to indicate an input to a pressure sensitive touchscreen by the user. The touchscreen and a respective controlling processor may be configured to recognize touchscreen gestures including, for example, but not limited to, tap, double tap, caliper gesture, drag and drop, slide, press and drag, hold and press, etc. The processor 1202, for example, may control a respective display device 1206 to provide visual representations of data captured by and/or received at the sensor data gathering device 1200. The visual representations may include still images and/or video images (e.g., animated images).

In some implementations, the output device(s) 1206 and/or the input device(s) 1208 include one or more wearable devices such as, in some examples, a heads-up display mounted onto eyeglasses, a face shield, a watch, and/or devices that may be integrated with other wearable communication devices, such as, for example, an ear bud or a Bluetooth®) hands free phone adaptor. The processor 1202 may control the output devices 1206 respectively, to provide information for the user. The information may include feedback (e.g., visible feedback, audible feedback, haptic feedback, textual feedback, numerical feedback, and graphical feedback) such as CPR feedback.

The one or more user input devices 1208 may include, for example, a keyboard, a mouse, joystick, trackball, or other pointing device, a microphone, a camera, etc. Further, the user input devices 1208 may be a touchscreen and/or another input/output device capable of providing information for the user and capturing information from the user. The touchscreen may be a pressure sensitive touchscreen.

In some implementations, one or more user input devices 1208 are configured to capture information, such as, for example, patient medical history (e.g., medical record information including age, gender, weight, body mass index, family history of heart disease, cardiac diagnosis, co-morbidity, left ventricular ejection fraction, medications, previous medical treatments, and/or other physiological information), physical examination results, patient identification, caregiver identification, healthcare facility information, etc.

The patient interface device(s) 1220 may include one or more therapy delivery component(s) 1222 and/or one or more sensor device(s) 1224. The therapy delivery component(s) 1222 are configured to deliver therapy to the patient and may be configured to couple to the patient. For example, the therapy delivery component(s) 1222 may include one or more of electrotherapy electrodes 1226 including defibrillation electrodes and/or pacing electrodes, chest compression devices (e.g., one or more belts or a piston) 1228, ventilation devices (e.g., a mask and/or tubes) 1230, intravenous devices 1232 (e.g., IBP probe), drug delivery devices, etc. The medical device, further, may include the one or more therapy delivery component(s) 1222 and/or may be configured to couple to the one or more therapy delivery component(s) 1222 in order to provide medical therapy to the patient. The therapy delivery component(s) 1222 may be configured to couple to the patient. For example, the caregiver may attach the electrodes 1226 to the patient and the medical device (e.g., a defibrillator or defibrillator/patient monitor) may provide electrotherapy to the patient via the electrotherapy electrodes 1226. These examples are not limiting of the disclosure as other types of medical devices, therapy delivery components, sensors, and therapy are within the scope of the disclosure.

The patient interface device(s) 1220 may include, incorporate, and/or be configured to couple to the one or more sensor(s) 1224 (e.g., the sensor 1032 of FIG. 10) which may be configured to couple to the patient. In various implementations, the sensor(s) 1224 may include one or more sensor devices configured to provide sensor data that includes, for example, but not limited to electrocardiogram (ECG), blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, images and/or videos via ultrasound, laryngoscopy, and/or other medical imaging techniques, 70 near-infrared reflectance spectroscopy, pneumography, cardiography, and/or patient movement. Images and/or videos may be two-dimensional or three-dimensional.

The sensor(s) 1224, in some implementations, are configured to provide signals indicative of sensor data to the device 1220. The sensor(s) 1224 may be configured to couple to the patient. For example, the sensor(s) 1224 may include cardiac sensing electrodes 1234, a chest compression sensor 1236, and/or ventilation sensors 1238. The cardiac sensing electrodes 1234 may be conductive and/or capacitive electrodes configured to measure changes in a patient's electrophysiology to measure the patient's ECG information. The sensing electrodes 1234 may further measure the transthoracic impedance and/or a heart rate of the patient.

The cardiac sensing electrodes 1234, in some implementations, are conductive and/or capacitive electrodes configured to measure changes in a patient's electrophysiology, for example to measure the patient's ECG information. For example, the sensing electrodes 1234 may be configured to measure the transthoracic impedance and/or a heart rate of the patient.

The ventilation sensors 1238 may include spirometry sensors, flow sensors, pressure sensors, oxygen and/or carbon dioxide sensors such as, for example, one or more of pulse oximetry sensors, oxygenation sensors (e.g., muscle oxygenation/pH), O2 gas sensors and capnography sensors, and combinations thereof.

The temperature sensors 1240 may include an infrared thermometer, a contact thermometer, a remote thermometer, a liquid crystal thermometer, a thermocouple, a thermistor, etc. and may measure patient temperature internally and/or externally.

The chest compression sensor 1236 may include one or more motion sensors including, for example, one or more accelerometers, one or more force sensors, one or more magnetic sensors, one or more velocity sensors, one or more displacement sensors, etc. The chest compression sensor 1236 may be, for example, but not limited to, a compression puck, a smartphone, a hand-held device, a wearable device, etc. The chest compression sensor 1236 may be configured to detect chest motion imparted by a rescuer and/or an automated chest compression device (e.g., a belt system, a piston system, etc.). The chest compression sensor 1236 may provide signals indicative of chest compression data including displacement data, velocity data, release velocity data, acceleration data, compression rate data, dwell time data, hold time data, blood flow data, blood pressure data, etc. In some embodiments, the sensing electrodes 1234 and/or the electrotherapy electrodes 1226 include or be configured to couple to the chest compression sensor 1236.

The one or more sensors 1224, in some implementations, generate signals indicative of physiological parameters of the patient. For example, the physiological parameters may include one or more of at least one vital sign, an ECG, blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, physical parameters as determined via ultrasound images, parameters determined via near-infrared reflectance spectroscopy, pneumography, and/or cardiography, etc. The ultrasound images may include ultrasound images of a patient's heart, carotid artery, and/or other components of the cardiovascular system. Additionally or alternatively, the one or more sensors 1224 may generate signals indicative of chest compression parameters, ventilation parameters, drug delivery parameters, fluid delivery parameters, etc.

In addition to delivering therapy to the patient, the therapy delivery component(s) 1222 may include, be coupled to, and/or function as sensors and provide signals indicative of sensor data to the device 1220. For example, the electrotherapy electrodes 1226 may be configured as cardiac sensing electrodes as well as electrotherapy delivery devices and may provide signals indicative of transthoracic impedance, electrocardiogram (ECG), heart rate and/or other physiological parameters. As another example, a therapeutic cooling device may be an intravenous cooling device. Such a cooling device may include an intravenous (IV) device 1232 as a therapy delivery component configured to deliver cooling therapy and sense the patient's temperature. For example, the IV device 1232 may be a catheter that includes saline balloons configured to adjust the patient's temperature via circulation of temperature controlled saline solution. In addition, the catheter may include a temperature probe configured to sense the patient's temperature (e.g., using a temperature sensor 1240). As a further example, an IV device 1232 may provide therapy via drug delivery and/or fluid management. The IV device 1232 may also monitor and/or enabling monitoring of a patient via blood sampling and/or venous pressure monitoring (e.g., central venous pressure (CVP) monitoring).

The sensor data gathering device 1200 may be configured to receive the sensor signals (e.g., from the therapy delivery component(s) 1222 and/or the sensor(s) 1224) and to process the sensor signals to determine and collect the patient data. The patient data may include patient data which may characterize a status and/or condition of the patient (e.g., physiological data such as electrocardiogramaart rate, respiration rate, temperature, glucose parameters, pulse oximetry, non-invasive hemoglobin parameters, capnography, oxygen saturation (SpO2), end tidal carbon dioxide (EtCO2), invasive blood pressure (IBP), non-invasive blood pressures (NIBP), tissue pH, tissue oxygenation, Near Infrared Spectroscopy (NIRS) measurements, etc.). Additionally or alternatively, the patient data may characterize the delivery of therapy (e.g., chest compression data such as compression depth, compression rate, etc.) and/or the patient data may characterize a status and/or condition of the medical equipment used to treat the patient (e.g., device data such as shock time, shock duration, attachment of electrodes, power-on, etc.).

In some implementations, the device sensor data gathering device 1200 is the medical device configured to deliver medical therapy to a patient. Thus, the sensor data gathering device 1200 may include a therapy delivery control module 1212. For example, the therapy delivery control module 1212 may be an electrotherapy delivery circuit that includes one or more capacitors configured to store electrical energy for a pacing pulse or a defibrillating pulse. The electrotherapy delivery circuit may further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 1212 may be a compression device electro-mechanical controller configured to control a mechanical compression device. As a further example, the therapy delivery control module 1212 may be an electro-mechanical controller configured to control drug delivery, temperature management, ventilation, and/or other type of therapy delivery. Alternatively, the sensor data gathering device 1200 may be configured to provide patient monitoring and/or diagnostic care without providing medical therapy.

Certain components 1202, 1204, 1206, 1208, 1210, 1212, and/or 1214 of the sensor data gathering device 1200, in some implementations, are communicatively coupled (directly and/or indirectly) to each other for bi-directional communication. Although shown as separate entities in FIG. 12, the one or more of the components of the sensor data gathering device 1200 may be combined into one or more discrete components and/or may be part of the processor 1202. The processor 1202 and the memory 1204 may include and/or be coupled to associated circuitry in order to perform the functions described herein.

The sensor data gathering device 1200, in some implementations, includes a patient interface device signal processor 1214. The patient interface device signal processor 1214 may include A/D converters and other hardware configured to receive and process signals from one or more of the patient interface devices 1220.

In various implementations, the sensor data gathering device 1200 is configured to couple to other computing devices. These other computing devices may be a medical device or a computing device (e.g., personal computer, a laptop computer, a mobile device, a hand-held device, a wireless device, a tablet computer, a wearable device such as a wrist-worn device, a head-worn device, heads up display, etc., or combinations thereof) adapted for medical use. The other medical devices may incorporate and/or be configured to couple to one or more patient interface device(s).

At least a portion of the processors as described herein, in some implementations, are physical processors (i.e., one or more integrated circuits configured to execute operations on a respective device as specified by software and/or firmware stored in a computer storage medium) operably coupled, respectively, to at least one memory device. The processors may be intelligent hardware devices (for example, but not limited to, a central processing unit (CPU), a graphics processing unit (GPU), one or more microprocessors, a controller or microcontroller, an application specific integrated circuit (ASIC), a digital signal processor (DSP), etc.) designed to perform the functions described herein and operable to carry out instructions on a respective device. Each of the processors may be one or more processors and may be implemented as a combination of hardware devices (e.g., a combination of DSP and a microprocessor, a set of microprocessors, one or more microprocessors in conjunction with a DSP core, or another such configuration). Each of the processors may include multiple separate physical entities that may be distributed in the data transfer cables and/or devices described herein.

At least a portion of the processors as described herein, in some implementations, are configured to execute processor-readable, processor-executable software code containing one or more instructions or code for controlling the processors to perform the functions as described herein. The processors may utilize various architectures including but not limited to a complex instruction set computer (CISC) processor, a reduced instruction set computer (RISC) processor, or a minimal instruction set computer (MISC). In various implementations, each processor may be a single-threaded or a multi-threaded processor. The processors may be, for example, but not limited to, an Intel® Itanium® or Itanium 2® processor(s), AMD® Opteron®, Athlon MP® processor(s), a Motorola® line of processor, or an ARM, Intel Pentium Mobile, Intel Core i5 Mobile, AMD A6 Series, AMD Phenom II Quad Core Mobile, or like devices.

The memories, as described herein, refer generally to a computer storage medium, including but not limited to RAM, ROM, FLASH, disc drives, fuse devices, and portable storage media, such as Universal Serial Bus (USB) flash drives, etc. Each of the memories may include, for example, random access memory (RAM), or another dynamic storage device(s) and may include read only memory (ROM) or another static storage device(s) such as programmable read only memory (PROM) chips for storing static information such as instructions for a coupled processor. Each memory may include USB flash drives that may store operating systems and other applications. The USB flash drives may include input/output components, such as a wireless transmitter and/or USB connector that can be inserted into a USB port of another computing device. Each memory may be long term and/or short term are not to be limited to a particular type of memory or number of memories, or type of media upon which memory is stored. Each memory includes a non-transitory processor-readable storage medium (or media) that stores the processor-readable, processor-executable software code. Each memory may store information and instructions. For example, each memory may include flash memory and/or another storage media may be used, including removable or dedicated memory in a mobile or portable device. As another example, hard disks such as the Adaptec® family of SCSI drives, an optical disc, an array of disks such as RAID (e.g., the Adaptec family of RAID drives), or another mass storage devices may be used. Each memory may include removable storage media such as, for example, external hard-drives, floppy drives, flash drives, zip drives, compact disc-read only memory (CD-ROM), compact disc-re-writable (CD-RW), or digital video disk-read only memory (DVD-ROM).

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

What is claimed is:

1. A data transfer cable for transferring sensor data between a sensor and a medical device, the data transfer cable comprising:
an insulative sheath surrounding a plurality of conductive wires;
a medical device connector for connecting the data transfer cable to a data port of a medical device;
at least one illumination element in electrical communication with at least one wire of the plurality of conductive wires, the at least one illumination element being configured to provide at least two visual indications visible in a location distal to the medical device connector, wherein
each visual indication of the at least two visual indications corresponds to a respective state of the data transfer cable or a sensor connected thereto; and
processing circuitry configured to
receive, from the medical device responsive to user interaction with a portion of a user interface of the medical device corresponding to the sensor connected to the data transfer cable, an identify signal, and
responsive to receiving the identify signal, cause a first illumination element of the at least one illumination element to provide a first visual indication of the at least two visual indications for indicating correspondence between the data transfer cable and the sensor connected thereto.

2. The data transfer cable of claim 1, further comprising an isolation device configured to limit current flow leakage between the medical device and the sensor.

3. The data transfer cable of claim 2, wherein the isolation device is configured to:
transfer power across an isolation barrier unidirectionally toward the processing circuitry; and
transmit communication signals bi-directionally across the isolation barrier.

4. The data transfer cable of claim 3, further comprising a noise shield disposed between the isolation device and the processing circuitry.

5. The data transfer cable of claim 1, wherein the sensor is one of an invasive blood pressure sensor, a non-invasive blood pressure sensor, a temperature sensor, a pulse oximetry sensor, a capnography sensor, or an airway flow sensor.

6. The data transfer cable of claim 1, wherein the sensor is one of a breath sounds sensor, a heart sounds sensor, a lung sounds sensor, a dual shock defibrillator sensor. an electroencephalogram (EEG) sensor, or a glucose monitoring sensor.

7. The data transfer cable of claim 1, wherein the sensor is an electrocardiogram sensor or an extended ECG sensor.

8. The data transfer cable of claim 7, wherein the at least one illumination element comprises a respective illumination element corresponding to each ECG contact of a plurality of ECG contacts configured for individual positioning upon a patient.

9. The data transfer cable of claim 8, wherein:
the user interaction comprises interaction with a selected ECG signal graph of a plurality of ECG signal graphs presented on a display of the medical device; and
causing the first illumination element to provide the first visual indication comprises causing illumination corresponding to a set of ECG contacts of the plurality of ECG contacts contributing to the selected ECG signal graph.

10. The data transfer cable of claim 1, further comprising a housing disposed along the insulative sheath or at a proximal end of the insulative sheath opposite the medical device connector, wherein the housing comprises the processing circuitry and the at least one illumination element.

11. The data transfer cable of claim 10, wherein the housing comprises the sensor.

12. The data transfer cable of claim 10, wherein the housing comprises, at an end opposite the insulative sheath, a sensor connector for releasably engaging a mating connector of a sensor device comprising the sensor.

13. The data transfer cable of claim 1, wherein:
the sensor comprises an airway flow sensor; and
the processing circuitry is configured to
receive, from the medical device, timing signals corresponding to delivery of airflow to a patient, and
using the timing signals, cause one or more illumination elements of the at least one illumination element to provide a second visual indication of the at least two visual indications for prompting airflow delivery by a caregiver.

14. The data transfer cable of claim 13, wherein a ventilation system comprising a bag valve mask comprises the airway flow sensor.

15. The data transfer cable of claim 13, wherein:
a ventilation system comprises or is connected with the one or more illumination elements; and
the one or more illumination elements are arranged such that the second visual indication can be recognized by a caregiver observing the one or more illumination elements from a plurality of orientations.

16. The data transfer cable of claim 15, wherein the one or more illumination elements are arranged on a three-dimensional protrusion connected to a housing of the ventilation system.

17. The data transfer cable of claim 15, wherein the one or more illumination elements are arranged on a pivoting attachment of the ventilation system.

18. The data transfer cable of claim 15, wherein the one or more illumination elements are arranged on a rotating attachment of the ventilation system.

19. The data transfer cable of claim 11, wherein:
the sensor comprises an airway flow sensor; and
the processing circuitry is configured to
receive, from the medical device, feedback signals corresponding to at least one of a timing and a volume of delivery of airflow to a patient, and
using the feedback signals, cause one or more illumination elements of the at least one illumination element to provide a second visual indication of the at least two visual indications for providing feedback to a caregiver regarding airflow delivery 20. The data transfer cable of claim 19, wherein the second visual indication visually mimics corresponding visual feedback presented in a region of a display of the medical device.

21. The data transfer cable of claim 19, wherein the second visual indication comprises at least one of a numeric rate indication or a numeric volume indication.

22. The data transfer cable of claim 10, wherein:
a digital display comprises the at least one illumination element; and
the housing comprises an aperture for the digital display.

23. The data transfer cable of claim 22, wherein the digital display is a liquid crystal display (LCD) or a light emitting diode (LED) display.

24. The data transfer cable of claim 10, wherein:
the at least one illumination element comprises at least one light emitting diode (LED); and
the housing comprises at least one translucent region disposed proximate each LED of the at least one LED.

25. The data transfer cable of claim 24, wherein:
a first LED of the at least one LED is a multi-color LED; and
the at least two visual indications comprise a first color indication of the multi-color LED and a second color indication of the multi-color LED.

26. The data transfer cable of claim 1, wherein the portion of the user interface of the medical device is a portion of a display of the medical device.

27. The data transfer cable of claim 26, wherein the display of the medical device is a touch display.

28. The data transfer cable of claim 1, wherein the data transfer cable comprises, at an end opposite the medical device connector, a sensor connector for releasably engaging a mating connector of a sensor device comprising the sensor.

29. The data transfer cable of claim 28, wherein the sensor connector is configured to releasably engage one of a set of sensor devices, each sensor device comprising a different type of sensor.

30. The data transfer cable of claim 29, wherein the processing circuitry is configured to format sensor data from the respective type of sensor of each sensor device of the set of sensor devices into a sensor-agnostic data format accepted by the medical device.

31. The data transfer cable of claim 1, wherein the processing circuitry is further configured to:
receive, from the medical device after connection of the medical device connector to the medical device, a connection signal; and
responsive to receiving the connection signal, cause one or more illumination elements of the at least one illumination element to provide a second visual indication of the at least two visual indications for indicating connection between the medical device and the data transfer cable.

32. The data transfer cable of claim 31, wherein the second visual indication remains illuminated while the data transfer cable is connected to the medical device.

33. The data transfer cable of claim 31, wherein the one or more illumination elements comprises the first illumination element.

34. The data transfer cable of claim 31, wherein the connection signal indicates that a data transfer connection with the data transfer cable has been authenticated by the medical device.

35. The data transfer cable of claim 34, wherein the processing circuitry is further configured to engage in an authentication handshake with the medical device.

36. The data transfer cable of claim 1, wherein the processing circuitry is further configured to:
receive at least one sensor signal from the sensor; and
responsive to receiving the at least one sensor signal, cause a second illumination element of the at least one illumination element to provide a second visual indication of the at least two visual indications for indicating data connection between the data transfer cable and the sensor.

37. The data transfer cable of claim 36, wherein the second illumination element is different than the first illumination element.

38. The data transfer cable of claim 36, wherein the second visual indication remains illuminated while the processing circuitry is communicating with the sensor.

39. The data transfer cable of claim 1, wherein:
the sensor comprises a cardiopulmonary resuscitation (CPR) compression sensor; and
the processing circuitry is configured to
receive, from the medical device, timing signals corresponding to delivery of compressions to a patient, and
using the timing signals, cause one or more illumination elements of the at least one illumination element to provide a second visual indication of the at least two visual indications for prompting compression delivery by a caregiver.

40. The data transfer cable of claim 1, wherein:
the sensor comprises a cardiopulmonary resuscitation (CPR) compression sensor; and
the processing circuitry is configured to
receive, from the medical device, feedback signals corresponding to at least one of a timing and a depth of compression delivery to a patient, and
using the feedback signals, cause one or more illumination elements of the at least one illumination element to provide a second visual indication of the at least two visual indications for providing feedback to a caregiver regarding compression delivery.

41. The data transfer cable of claim 40, wherein the second visual indication visually mimics corresponding visual feedback presented in a region of a display of the medical device.

42. The data transfer cable of claim 40, wherein the second visual indication comprises at least one of a numeric rate indication or a numeric depth indication.

43. The data transfer cable of claim 1, wherein:
the sensor is an invasive blood pressure (IBP) sensor; and
the processing circuitry is configured to
receive, from the medical device, a zeroing signal corresponding to zeroing an IBP probe comprising the IBP sensor, and
responsive to the zeroing signal, cause one or more illumination elements of the at least one illumination element to provide a second visual indication of the at least two visual indications for identifying the IBP probe is being zeroed.

44. The data transfer cable of claim 43, wherein the processing circuitry is configured to cause one or more illumination elements of the at least one illumination element to provide a third visual indication of the at least two visual indications for prompting a user to zero the IBP probe.

45. A patient monitoring and treatment system for providing sensor data capture capabilities, the system comprising:
a medical device comprising
a display,
at least one data interface port configured to enable power transfer to a sensor unit and data communications between at least one sensor of the sensor unit and the medical device, and
medical device processing circuitry configured to analyze sensor signals received via the at least one data interface port and present information corresponding to the sensor signals upon the display; and
a data transfer cable configured to matingly connect to the at least one data interface port, the data transfer cable comprising
an insulative sheath surrounding a plurality of conductive wires,
a medical device connector for connecting the data transfer cable to a given data interface port of the at least one data interface port of the medical device,
at least one illumination element in electrical communication with at least one wire of the plurality of conductive wires, the at least one illumination element disposed in a location distal to the medical device connector, and
cable processing circuitry configured to enable communication between the medical device and the sensor unit and to enable feedback to a user via the at least one illumination element;
wherein the medical device processing circuitry is further configured to coordinate visual feedback presentations to the user via the display of the medical device and the at least one illumination element of the data transfer cable.

46. The system of claim 45, wherein coordinating visual feedback presentations comprises causing presentation, at both the display of the medical device and at the at least one illumination element, of coaching feedback for using the sensor unit.

47. The system of claim 46, wherein the coaching feedback visually mimics corresponding visual feedback presented in a region of the display of the medical device.

48. The system of claim 46, wherein:
the sensor unit is an invasive blood pressure (IBP) probe comprising an IBP sensor; and
the coaching feedback comprises feedback for setting up the IBP probe.

49. The system of claim 48, wherein coordinating the visual feedback presentations comprises:
presenting, on the display, a prompt for zeroing the IBP probe; and
providing, to the medical device processing circuitry, instructions to provide visual feedback, via the at least one illumination element, corresponding to initiation of zeroing the IBP probe.

50. The system of claim 48, wherein coordinating the visual feedback presentations comprises:
presenting, on the display, a prompt for identifying a use case for the IBP probe; and
providing, to the cable processing circuitry, instructions to provide visual feedback, via the at least one illumination element, corresponding to selection of the use case for the IBP probe.

51. The system of claim 50, wherein the use case is one of invasive blood pressure (IPB), arterial blood pressure (ART), pulmonary artery pressure (PAP), central venous pressure (CVP), or intra-cranial pressure (ICP).

52. The system of claim 46, wherein:
the sensor unit is a ventilation unit comprising an airflow sensor; and
the coaching feedback comprises feedback for delivering a target volume of air to a patient.

53. The system of claim 52, wherein the cable processing circuitry is configured to:
receive, from the medical device, timing signals corresponding to delivery of airflow to a patient; and
using the timing signals, cause one or more illumination elements of the at least one illumination element to present the coaching feedback for airflow delivery by a caregiver.

54. The system of claim 52, wherein a bag valve mask comprises the at least one illumination element.

55. The system of claim 54, wherein the at least one illumination element is arranged such that the coaching feedback can be recognized by a caregiver observing the bag valve mask from a plurality of orientations.

56. The system of claim 54, wherein the at least one illumination element is arranged on a three-dimensional protrusion of the bag valve mask.

57. The system of claim 54, wherein the at least one illumination element is arranged on a pivoting attachment of the bag valve mask.

58. The system of claim 54, wherein the at least one illumination element is arranged on a rotating attachment of the bag valve mask.

59. The system of claim 52, wherein the coaching feedback comprises at least one of a numeric rate indication or a numeric volume indication.

60. The system of claim 52, further comprising a second data transfer cable comprising:
a second insulative sheath surrounding a plurality of second conductive wires;
a second medical device connector for connecting the second data transfer cable to another given data interface port of the at least one data interface port of the medical device;
at least one second illumination element in electrical communication with at least one wire of the plurality of second conductive wires, the at least one second illumination element being visible in a location distal to the second medical device connector; and
second cable processing circuitry configured to enable communication between the medical device and a chest compression sensor unit and to enable feedback to a second user via the at least one second illumination element;
wherein the medical device processing circuitry is configured to coordinate ventilation feedback to the user and compression feedback to the second user via the at least one illumination element of the data transfer cable and the at least one second illumination element of the second data transfer cable.

61. The system of claim 60, wherein coordinating the ventilation feedback and the compression feedback comprises coordinating prompting for ventilation timing with chest compression timing.

62. The system of claim 46, wherein:
the sensor comprises a cardiopulmonary resuscitation (CPR) compression sensor; and
the cable processing circuitry is configured to
receive, from the medical device, timing signals corresponding to delivery of compressions to a patient, and
using the timing signals, cause one or more illumination elements of the at least one illumination element to present the coaching feedback for prompting compression delivery by a caregiver.

63. The system of claim 61, wherein the cable processing circuitry is configured to:
receive, from the medical device, feedback signals corresponding to at least one of a timing or a depth of compression delivery to a patient, and
using the feedback signals, cause one or more illumination elements of the at least one illumination element to present a level of sufficiency of the at least one of the timing and the depth of the compression delivery.

64. The system of claim 63, wherein presenting the level of sufficiency comprises presenting at least one of a numeric rate indication or a numeric depth indication.

65. The system of claim 63, further comprising a second data transfer cable comprising:
a second insulative sheath surrounding a plurality of second conductive wires;
a second medical device connector for connecting the second data transfer cable to another given data interface port of the at least one data interface port of the medical device;
at least one second illumination element in electrical communication with at least one wire of the plurality of second conductive wires, the at least one second illumination element being visible in a location distal to the second medical device connector; and
second cable processing circuitry configured to enable communication between the medical device and a ventilation sensor unit and to enable feedback to a second user via the at least one second illumination element;
wherein the medical device processing circuitry is configured to coordinate CPR feedback to the user and ventilation feedback to the second user via the at least one illumination element of the data transfer cable and the at least one second illumination element of the second data transfer cable.

66. The system of claim 65, wherein coordinating the CPR feedback and the ventilation feedback comprises coordinating prompting for chest compression timing with ventilation timing.

67. The system of claim 45, wherein:
the display comprises a touch-sensitive interface; and
the medical device comprises a lock control for disabling the touch-sensitive interface.

68. The system of claim 67, wherein, responsive to disabling the touch-sensitive interface via actuation of the lock control, the medical device processing circuitry is configured to present, upon the display, a lock enabled indicator.

69. The system of claim 67, wherein the medical device comprises a manual navigation control for navigating and interacting with contents of the display.

70. The system of claim 69, wherein navigating and interacting via the manual navigation control is disabled while the lock control is in a disabled position.

71. A ventilation sensor unit comprising:
a housing comprising an airflow sensor;
an airflow delivery element for manually controlling airflow delivery to a patient;
a data transfer cable extending from the housing, the data transfer cable comprising an insulative sheath surrounding a plurality of conductive wires, and
a medical device connector for connecting the data transfer cable to a data port of a medical device;
at least one illumination element in electrical communication with at least one wire of the plurality of conductive wires; and
processing circuitry configured to
receive, from the medical device, timing signals corresponding to delivery of airflow to a patient,
using the timing signals, cause one or more illumination elements of the at least one illumination element to provide a visual indication for prompting airflow delivery by a caregiver, and
provide, to the medical device via the data transfer cable, at least one airflow signal from the airflow sensor indicative of at least one of a rate or a volume of airflow delivered to the patient.

72. The ventilation sensor unit of claim 71, wherein a bag valve mask comprises the housing.

73. The ventilation sensor unit of claim 71, wherein the one or more illumination elements are arranged such that the visual indication can be recognized by a caregiver observing the housing from a plurality of orientations.

74. The ventilation sensor unit of claim 71, wherein the one or more illumination elements

75. The ventilation sensor unit of claim 74, wherein the one or more illumination elements comprise a plurality of LED elements arranged in a ring formation on the housing.

76. The ventilation sensor unit of claim 75, wherein the plurality of LED elements comprise multi-colored LED elements.

77. The ventilation sensor unit of claim 74, wherein the one or more illumination elements are arranged to provide a digital display configured to present numeric feedback to the caregiver.

78. The ventilation sensor unit of claim 77, wherein the numeric feedback comprises at least one of a volume or a rate.

79. The ventilation sensor unit of claim 71, wherein the one or more illumination elements are arranged on a three-dimensional protrusion of the housing.

80. The ventilation sensor unit of claim 71, wherein the one or more illumination elements are arranged on a pivoting attachment of the housing.

81. The ventilation sensor unit of claim 71, wherein the one or more illumination elements are arranged on a rotating attachment of the housing.

82. The ventilation sensor unit of claim 71, wherein the processing circuitry is further configured to:
receive, from the medical device, feedback signals corresponding to at least one of a timing and a volume of recent delivery of airflow to a patient; and
using the feedback signals, cause one or more illumination elements of the at least one illumination element to provide sufficiency feedback to a caregiver regarding sufficiency of airflow delivery.

83. The ventilation sensor unit of claim 82, wherein the sufficiency feedback visually mimics corresponding visual feedback presented in a region of a display of the medical device.

84. The ventilation sensor unit of claim 82, wherein the sufficiency feedback comprises at least one of a numeric rate indication or a numeric volume indication.

85. The ventilation sensor unit of claim 82, wherein the sufficiency feedback comprises a respective color of a set of colors corresponding to a target range and outside of the target range.

86. A system for monitoring invasive blood pressure (IBP) in a patient, the system comprising:
an invasive blood pressure (IBP) probe comprising
a housing, and
an invasive blood pressure (IBP) sensor;
a data transfer cable extending from the IBP probe, the data transfer cable comprising an insulative sheath surrounding a plurality of conductive wires, and
a medical device connector for connecting the data transfer cable to a data port of a medical device; and
the medical device comprising
the data port,
a display, and
processing circuitry configured to
recognize insertion of the data transfer cable in the data port,
present, on the display, a prompt for zeroing the IBP probe, and
responsive to input to the medical device by a caregiver, initiate a zeroing process with the IBP probe.

87. The system of claim 86, wherein the processing circuitry of the medical device is configured to:
present, to the caregiver, a prompt on the display of the medical device to one of a set of use cases for the IBP probe.

88. The system of claim 87, wherein the set of use cases comprises two or more of invasive blood pressure (IBP), arterial blood pressure (ART), pulmonary artery pressure (PAP), central venous pressure (CVP), or intra-cranial pressure (ICP).

89. The system of claim 87, wherein, responsive to the caregiver entering a selected use case of the set of use cases, the processing circuitry of the medical device formats a section of the display to present metrics related to the IBP probe in a format corresponding to the selected use case.

90. The system of claim 86, wherein the IBP probe further comprises:
at least one illumination element in electrical communication with at least one wire of the plurality of conductive wires; and
probe processing circuitry configured to cause presentation of visual feedback to the caregiver via the at least one illumination element.

91. The system of claim 90, wherein the processing circuitry of the medical device is configured to:
detect interaction with a region of the display presenting metrics related to the IBP probe; and
responsive to the detecting, issue a signal via to the probe processing circuitry of the IBP probe to cause illumination of one or more illumination elements of the at least one illumination element.

92. The system of claim 90, wherein the processing circuitry of the medical device is further configured to provide, to the probe processing circuitry, instructions to provide visual feedback, via the at least one illumination element, corresponding to initiation of zeroing the IBP probe.

93. The system of claim 86, wherein:
the IBP probe further comprises probe processing circuitry configured to provide authentication information to the processing circuitry of the medical device; and
the processing circuitry of the medical device is configured, after recognizing insertion of the data transfer cable in the data port, initiate an authentication sequence with the probe processing circuitry.

94. The system of claim 86, wherein:
the data transfer cable further comprises data transfer cable processing circuitry configured to provide authentication information to the processing circuitry of the medical device; and
the processing circuitry of the medical device is configured to, after recognizing insertion of the data transfer cable in the data port, initiate an authentication sequence with the data transfer cable processing circuitry.

95. The system of claim 94, wherein the IBP probe is releasably attachable to the data transfer cable.

96. The system of claim 95, wherein the IBP probe is one type of a plurality of types of sensor devices compatible to releasably attach to the data transfer cable.

97. The system of claim 86, wherein the data transfer cable further comprises:
at least one illumination element in electrical communication with at least one wire of the plurality of conductive wires; and
data transfer cable processing circuitry configured to cause presentation of visual feedback to the caregiver via the at least one illumination element.

98. The system of claim 97, wherein the processing circuitry of the medical device is further configured to provide, to the data transfer cable processing circuitry, instructions to provide visual feedback, via the at least one illumination element, corresponding to initiation of zeroing the IBP probe.

99. The system of claim 97, wherein the processing circuitry of the data transfer cable is further configured to:
receive, from the medical device, a zeroing signal corresponding to zeroing an IBP probe comprising the IBP sensor; and
responsive to the zeroing signal, cause one or more illumination elements of the at least one illumination element to provide the visual feedback for identifying the IBP probe is being zeroed.

100. The system of claim 97, wherein the IBP probe is releasably attachable to the data transfer cable.

101. The system of claim 100, wherein the IBP probe is one type of a plurality of types of sensor devices compatible to releasably attach to the data transfer cable.
